(19) United States
(12) Patent Application Publication
(10) Pub. No.: US 2017/0348232 A1
(43) Pub. Date: Dec. 7, 2017

(54) FORMULATIONS OF GABOXADOL FOR TREATMENT OF ANGELMAN SYNDROME, FRAGILE X SYNDROME AND FRAGILE X-ASSOCIATED TREMOR/ATAXIA SYNDROME

(71) Applicant: Ovid Therapeutics Inc., New York, NY (US)

(72) Inventor: Matthew DURING, Weston, CT (US)

(21) Appl. No.: 15/616,460

(22) Filed: Jun. 7, 2017

Related U.S. Application Data
(60) Provisional application No. 62/346,763, filed on Jun. 7, 2016.

(51) Int. Cl. A61K 9/00; A61K 9/20; A61K 9/48; A61K 31/437

(52) U.S. Cl. CPC A61K 9/0056; A61K 31/437; A61K 9/20; A61K 9/48

(57) ABSTRACT

Methods and pharmaceutical compositions for treating Angelman syndrome, Fragile X syndrome and Fragile X-associated tremor/ataxia syndrome with gaboxadol or a pharmaceutically acceptable salt thereof are provided.

### TECHNICAL FIELD Methods of treating Angelman syndrome, Fragile X syndrome and Fragile X-associated tremor/ataxia syndrome with gaboxadol or a pharmaceutically acceptable salt thereof.

### BACKGROUND

Gaboxadol (4,5,6,7-tetrahydroisoxazolo [5,4-c]pyridine-3-ol) (THIP)) is described in EP Patent No. 0000338 and in EP Patent No. 0840601, U.S. Pat. Nos. 4,278,676, 4,362,731, 4,353,910, and WO 2005/094820. Gaboxadol is a selective GABA_{A} receptor agonist with a preference for δ-subunit containing GABA_{A} receptors. In the early 1980s gaboxadol was the subject of a series of pilot studies that tested its efficacy as an analgesic and anxiolytic, as well as a treatment for tardive dyskinesia, Huntington's disease, Alzheimer's disease, and spasticity. In the 1990s gaboxadol moved into late stage development for the treatment of insomnia. The development was discontinued after the compound failed to show significant effects in sleep onset and sleep maintenance in a three-month efficacy study. Additionally, patients with a history of drug abuse who received gaboxadol experienced a steep increase in psychiatric adverse events.

Treatments for disorders such as Angelman syndrome, Fragile X syndrome and Fragile X-associated tremor/ataxia syndrome are limited. Angelman syndrome is a neurodevelopmental disorder caused by loss of function of the UBE3A gene encoding a ubiquitin E3 ligase. Motor dysfunction is a characteristic feature of Angelman syndrome, but neither the mechanisms of action nor effective therapeutic strategies have yet been elucidated. Administering low doses of gaboxadol has been shown to improve the abnormal firing properties of a population of Purkinje cells in cerebellar brain slices and reduces cerebellar ataxia in Ube3α-deficient mice in vivo. These results suggest that pharmacologically increasing tonic inhibition may be a useful strategy for alleviating motor dysfunction in Angelman syndrome. Egawa, et al., Science Translational Medicine, 4:163ra157 (2012).

Fragile X syndrome (FXS) may be the most common genetic cause of intellectual disability and the most common single-gene cause of autism. It is caused by mutations on the fragile X mental retardation gene (FMR1) and lack of fragile X mental retardation protein, which in turn, leads to decreased inhibition of translation of many synaptic proteins. The main efforts have focused on metabotropic glutamate receptor (mGluR) targeted treatments; however, investigation on the gamma-aminobutyric acid (GABA) system and its potential as a targeted treatment is less emphasized. Fragile X mouse models show decreased GABA subunit receptors, decreased synthesis of GABA, increased catabolism of GABA, and overall decreased GABAergic input in many regions of the brain. These symptoms are also observed in individuals with autism and other neurodevelopmental disorders, therefore targeted treatments for Fragile X syndrome are leading the way in the treatment of other neurodevelopmental syndromes and autism. Potential GABAergic treatments, such as riluzole, gaboxadol, tiagabine, and vigabatrin have been discussed. However, further studies are needed to determine the safety and efficacy of GABAergic treatments for Fragile X syndrome. Moreover, further studies in fragile X animal models are necessary to provide cumulative evidence in the efficacy and safety of gaboxadol. Lozano et al., Neuropsychiatr Dis Treat., 10: 1769-1779 (2014).

Fragile X-associated tremor/ataxia syndrome (FXTAS) is a late-onset disorder, usually occurring after age 50. Mutations in the FMR1 gene increase the risk of developing FXTAS. The mutation relates to a DNA segment known as a CGG triplet repeat which is expanded within the FMR1 gene. Normally, this DNA segment is repeated from 5 to about 40 times. In people with FXTAS the CGG segment may be repeated 55 to 200 times. This mutation is known as an FMR1 gene premutation. An expansion of more than 200 repeats, a full mutation, causes Fragile X syndrome discussed above. FXTAS is typically characterized by problems with movement and thinking ability (cognition). FXTAS signs and symptoms usually worsen with age. Affected individuals have areas of damage in the cerebellum, the area of the brain that controls movement. Characteristic features of FXTAS are intention tremor, which is trembling or shaking of a limb when trying to perform a voluntary movement such as reaching for an object, and problems with coordination and balance (ataxia). Many affected individuals develop other movement problems, such as parkinsonism, which includes tremors when not moving (resting tremor), rigidity, and unusually slow movement (bradykinesia). In addition, affected individuals may have reduced sensation, numbness or tingling, pain, or muscle weakness in the lower limbs, and inability to control the bladder or bowel. Other symptoms may include chronic pain syndromes, such as fibromyalgia and chronic migraine, hypothyroidism, hypertension, sleep apnea, vertigo, olfactory dysfunction, and hearing loss. People with FXTAS commonly have cognitive disabilities such as short-term memory loss and loss of executive function, which is the ability to plan and implement actions and develop problem-solving strategies. Loss of this function impairs skills such as impulse control, self-monitoring, focusing attention appropriately, and cognitive flexibility. Many people with FXTAS experience psychiatric symptoms such as anxiety, depression, moodiness, or irritability.

There is currently no targeted therapeutic intervention that can arrest or reverse the pathogenesis of FXTAS. However a number of treatment approaches of potential symptomatic benefit have been suggested. Primidone, beta-blockers such as propanolol, topiramate, carbidopa/levodopa, and benzodiazepines have been suggested to control tremors associated with FXTAS; botulinum toxin for involuntary muscle activities, such as dystonia and spasticity; carbidopa/levodopa, amantadine and buspirone for ataxia; cholinesterase inhibitors such as donepezil, and memantine (an NMDA antagonist) for cognitive deficits and dementia; and antidepressants and antipsychotics for psychiatric symptoms. See, e.g., Hagerman, et al., Clin Intery Aging. 2008 June; 3 (2): 251-262.

There remains a need for effective treatments of patients with Angelman syndrome, Fragile X syndrome, and Fragile X-associated tremor/ataxia syndrome.

### SUMMARY

A method of treating a disorder selected from the group consisting of Angelman syndrome, Fragile X syndrome and Fragile X-associated tremor/ataxia syndrome is provided which includes administering to a patient in need thereof a pharmaceutical composition comprising about 0.05 mg to about 100 mg gaboxadol or a pharmaceutically acceptable salt thereof wherein the composition provides a Tₘₐₓ of less than 20 minutes. In embodiments, the composition is a modified release dosage form. In embodiments, the modified release dosage form includes an orally disintegrating dosage form. In embodiments, the modified release dosage form includes an extended release dosage form. In embodiments, the modified release dosage form includes a delayed release dosage form. In embodiments, the modified release dosage form includes a pulsatile release dosage form. In embodiments, the composition provides a Cₘₐₓ of less than 400 ng/ml. In embodiments, the method provides the amount of gaboxadol or a pharmaceutically acceptable salt thereof within the patient 4 hours after administration of the pharmaceutical composition is between about 65% to about 85% less than the administered dose. In embodiments, the method provides the amount of gaboxadol or a pharmaceutically acceptable salt thereof within the patient 4 hours after administration of the pharmaceutical composition is more than 50% of the administered dose. In embodiments, the method provides improvement in at least one of the following symptoms: tremors, rigidity, ataxia, bradykinesia, gait, speech impairment, vocalization difficulties, cognition impairment, impaired motor activity, clinical seizure, hypotonia, hypertonia, feeding difficulty, drooling, mouthing behavior, sleep difficulties, hand flapping, easily provoked laughter, short attention span, reduced sensation, numbness or tingling, pain, muscle weakness in the lower limbs, inability to control the bladder or bowel, chronic pain syndromes, fibromyalgia, migraine, hypothyroidism, hypertension, sleep apnea, vertigo, olfactory dysfunction, hearing loss, short-term memory loss, loss of executive function, impulse control difficulties, self-monitoring difficulties, attention focusing difficulties, cognitive inflexibility, anxiety, depression, moodiness, irritability.

A pharmaceutical dosage form is provided which includes a therapeutically effective amount of gaboxadol or a pharmaceutically acceptable salt thereof, and at least one pharmaceutically acceptable excipient, wherein the dosage form provides a Tₘₐₓ of less than about 20 minutes and a Cₘₐₓ greater than about 100 ng/mL. In embodiments, the Cₘₐₓ is less than about 400 ng/ml. In embodiments, the dosage form is a modified release dosage form. In embodiments, the modified release dosage form includes an orally disintegrating dosage form. In embodiments, the modified release dosage form includes an extended release dosage form. In embodiments, the modified release dosage form includes a delayed release dosage form. In embodiments, the modified release dosage form includes a pulsatile release dosage form. In embodiments, the amount of gaboxadol or a pharmaceutically acceptable salt thereof ranges from 0.05 mg to 100 mg. In embodiments, the dosage form provides delivery of gaboxadol or a pharmaceutically acceptable salt thereof such that the amount of gaboxadol or a pharmaceutically acceptable salt thereof within the patient 4 hours after administration of the pharmaceutical composition is between about 65% to about 85% less than the administered dose. In embodiments, the dosage form provides delivery of gaboxadol or a pharmaceutically acceptable salt thereof such that the amount of gaboxadol or pharmaceutically acceptable salt thereof within the patient 4 hours after administration of the pharmaceutical composition is more than 50% of the administered dose.

### BRIEF DESCRIPTION OF THE DRAWINGS

FIG. 1 shows mean plasma concentration-time profiles of gaboxadol following single oral doses (2.5, 5, 10, 15, and 20 mg).

FIG. 2 shows plasma concentration-time profiles for a single administration of four gaboxadol 2.5 mg fast modified release film coated tablets, a single administration of a gaboxadol 10 mg slow modified release film coated tablet and a single administration of a gaboxadol 10 mg conventional capsule.

FIG. 3 shows the mean plasma concentrations of gaboxadol following administration of the orally disintegrating tablets and monohydrate capsule formulations.

### DETAILED DESCRIPTION

Described herein are compositions and methods for treating Angelman syndrome, Fragile X syndrome and/or Fragile X-associated tremor/ataxia syndrome by administering to a patient in need thereof a modified release pharmaceutical composition including gaboxadol or a pharmaceutically acceptable salt thereof. Many pharmaceutical products are administered as a fixed dose, at regular intervals, to achieve therapeutic efficacy. The duration of action is typically reflected by plasma half-life of the drug post administration. Gaboxadol has a relatively short half-life (t^{1/2}=1.5 h). Since efficacy is often dependent on rapid onset of action and sufficient exposure within the central nervous system administration of CNS drugs with a short half-life may require frequent maintenance dosing.

In embodiments, pharmaceutical compositions herein provide modified release of gaboxadol or a pharmaceutically acceptable salt thereof resulting in pharmacokinetic properties which include a Tₘₐₓ of 20 minutes or less. Accordingly, dosage forms are described that provide a rapid onset of action. In embodiments, pharmaceutical compositions having modified release profiles provide pharmacokinetic properties which result in both rapid onset and sustained duration of action. In embodiments, pharmaceutical compositions having modified release profiles provide pharmacokinetic properties which result in both rapid onset and extended release. In embodiments, pharmaceutical compositions having modified release profiles provide pharmacokinetic properties which result in both rapid onset and delayed release. In embodiments, pharmaceutical compositions having modified release profiles provide pharmacokinetic properties which result in both rapid onset and extended release which is pulsatile in nature. In embodiments, pharmaceutical compositions having modified release profiles provide pharmacokinetic properties which result in both rapid onset and delayed release which is pulsatile in nature. In embodiments, pharmaceutical compositions having modified release profiles provide pharmacokinetic properties which result in a combination of rapid onset, delayed release and sustained duration of action. In embodiments, pharmaceutical compositions having modified release profiles provide pharmacokinetic properties which result in a combination of rapid onset, delayed release and sustained duration of action which is pulsatile in nature.

Conventional (or unmodified) release oral dosage forms such as tablets or capsules typically release medications into the stomach or intestines as the tablet or capsule shell dissolves. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

In embodiments, pharmaceutical compositions herein provide immediate release of gaboxadol or a pharmaceutically acceptable salt thereof resulting in pharmacokinetic properties which include a Tₘₐₓ of 20 minutes or less. In embodiments, pharmaceutical compositions herein provide a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. Such pharmaceutical compositions include ODDFs such as orally disintegrating tablets (ODTs).

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Patients with Angelman syndrome, Fragile X syndrome or Fragile X-associated tremor/ataxia syndrome may exhibit such behavior. In addition, ODDFs herein provide a rapid onset of action which can provide rapid alleviation or cessation of symptoms associated with Angelman syndrome, Fragile X syndrome or Fragile X-associated tremor/ataxia syndrome. In embodiments, when administered to an oral cavity, an ODDF herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds.

An ODT is a solid dosage form containing a medicinal substance or active ingredient which disintegrates rapidly, usually within a matter of seconds when placed upon the tongue. The disintegration time for ODTs generally ranges from several seconds to about a minute. ODTs are designed to disintegrate or dissolve rapidly on contact with saliva, thus eliminating the need to chew the tablet, swallow the intact tablet, or take the tablet with liquids. As with ODDFs in general, this mode of administration can be beneficial to people who may have problems swallowing tablets whether it be from physical infirmity or psychiatric in nature. Patients with Angelman syndrome, Fragile X syndrome or Fragile X-associated tremor/ataxia syndrome may exhibit such behavior. In addition, ODTs herein provide a rapid onset of action which can result in a rapid alleviation or cessation of symptoms associated with Angelman syndrome, Fragile X syndrome or Fragile X-associated tremor/ataxia syndrome. In embodiments, an ODT herein disintegrates in less than one minute, less than 55 seconds, less than 50 seconds, less than 45 seconds, less than 40 seconds, less than 35 seconds, less than 30 seconds, less than 25 seconds, less than 20 seconds, less than 15 seconds, less than 10 seconds, or less than 5 seconds, based upon, e.g., the United States Pharmacopeia (USP) disintegration test method set forth at section 701, Revision Bulletin Official Aug. 1, 2008.

In embodiments, the fast dissolving property of the ODTs requires quick ingress of water into the tablet matrix. This may be accomplished by maximizing the porous structure of the tablet, incorporation of suitable disintegrating agents and use of highly water-soluble excipients in the formulation. Excipients used in ODTs typically contain at least one superdisintegrant (which can have a mechanism of wicking, swelling or both), a diluent, a lubricant and optionally a swelling agent, sweeteners and flavorings. See, e.g., Nagar et al., Journal of Applied Pharmaceutical Science, 2011; 01 (04):35-45, incorporated herein by reference. Superdisintegrants can be classified as synthetic, natural and co-processed. In this context synthetic superdisintegrants can be exemplified by sodium starch glycolate, croscarmellose sodium, cross-linked polyvinylpyrrolidone, low-substituted hydroxypropyl cellulose, microcrystalline cellulose, partially pregelatinized starch, cross-linked alginic acid and modified resin. Natural superdisintegrants can be processed mucilages and gums are obtained from plants and can be exemplified by Lepidium sativum seed mucilage, banana powder, gellan gum, locust bean gum, xanthan gum, guar gum, gum karaya, cassia fistula seed gum, mangifera indica gum, carrageenan, agar from Gelidium amansii and other red algaes, soy polysaccharide and chitosan. Diluents can include, e.g., mannitol, sorbitol, xylitol, calcium carbonate, magnesium carbonate, calcium sulfate, magnesium trisilicate and the like. Lubricants can include, e.g., magnesium stearate and the like. Those skilled in the art are familiar with ODT manufacturing techniques.

Other ODDFs which may be used herein include rapidly dissolving films which are thin oral strips that release medication such as gaboxadol or a pharmaceutically acceptable salt thereof quickly after administration to the oral cavity. The film is placed on a patient's tongue or any other mucosal surface and is instantly wet by saliva whereupon the film rapidly hydrates and dissolves to release the medication. See. e.g., Chaturvedi et al., Curr Drug Deliv. 2011 July; 8 (4):373-80. Fastcaps are a rapidly disintegrating drug delivery system based on gelatin capsules. In contrast to conventional hard gelatin capsules, fastcaps consist of a gelation of low bloom strength and various additives to improve the mechanical and dissolution properties of the capsule shell. See, e.g., Ciper and Bodmeier, Int J Pharm. 2005 Oct. 13; 303 (1-2):62-71. Freeze dried (lyophilized) wafers are rapidly disintegrating, thin matrixes that contain a medicinal agent. The wafer or film disintegrates rapidly in the oral cavity and releases drug which dissolves or disperses in the saliva. See, e.g., Boateng et al., Int J Pharm. 2010 Apr. 15; 389 (1-2):24-31. Those skilled in the art are familiar with various techniques utilized to manufacture ODDFs such as freeze drying, spray drying, phase transition processing, melt granulation, sublimation, mass extrusion, cotton candy processing, direct compression, etc. See, e.g., Nagar et al., supra.

When administered, ODDFs containing gaboxadol or a pharmaceutically acceptable salt thereof disintegrate rapidly to release the drug, which dissolves or disperses in the saliva. The drug may be absorbed in the oral cavity, e.g., sublingually, buccally, from the pharynx and esophagus or from other sections of gastrointestinal tract as the saliva travels down. In such cases, bioavailability can be significantly greater than that observed from conventional tablet dosage forms which travel to the stomach or intestines where drug can be released.

ODDFs herein provide a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, the amount of gaboxadol or pharmaceutically acceptable salt thereof within the patient about 4 hours after administration of the pharmaceutical composition is between about 65% to about 85% less than the administered dose. In embodiments, the amount of gaboxadol or pharmaceutically acceptable salt thereof within the patient about 4 hours after administration of the pharmaceutical composition is less than 65% 70%, 75%, 80%, or 85% of the administered dose.

In embodiments, ODDFs herein provide an in vivo plasma profile having Cₘₐₓ less than about 2500 ng/ml, 2000 ng/ml, 1750 ng/ml, 1500 ng/ml, 1250 ng/ml, 1000 ng/ml, 750 ng/ml, 500 ng/ml, 450 ng/ml, 400 ng/ml, 350 ng/ml, 300 ng/ml, 250 ng/ml, 200 ng/ml, 150 ng/ml, 100 ng/ml, 50 ng/ml or 25 ng/ml. In embodiments, ODDFs herein provide an in vivo plasma profile having a AUC_{0-∞}, of less than about, e.g., 900 ng·hr/ml, 850 ng·hr/ml, 800 ng·hr/ml, 750 ng·hr/ml, or 700 ng·hr/ml 650 ng·hr/ml, 600 ng·hr/ml, 550 ng·hr/ml, 500 ng·hr/ml, or 450 ng·hr/ml. In embodiments, ODDFs herein provide an in vivo plasma profile having a AUC_{0-∞} of less than about, e.g., 400 ng·hr/ml, 350 ng·hr/ml, 300 ng·hr/ml, 250 ng·hr/ml, or 200 ng·hr/ml. In embodiments, ODDFs herein provide an in vivo plasma profile having a AUC_{0-∞} of less than about, e.g., 150 ng·hr/ml, 100 ng·hr/ml, 75 ng·hr/ml, or 50 ng·hr/ml.

In embodiments, pharmaceutical compositions having modified release profiles provide pharmacokinetic properties which result in both rapid onset and sustained duration of action. Such pharmaceutical compositions include an immediate release aspect and an extended release aspect. Immediate release aspects are discussed above in connection with ODDFs. Extended release dosage forms (ERDFs) have an extended release profiles and are those that allow a reduction in dosing frequency as compared to that presented by a conventional dosage form, e.g., a solution or unmodified release dosage form. ERDFs provide a sustained duration of action of a drug. In embodiments, modified release dosage forms herein incorporate an ODDF aspect to provide immediate release of a loading dose and then an ERDF aspect that provides prolonged delivery to maintain drug levels in the blood within a desired therapeutic range for a desirable period of time in excess of the activity resulting from a single dose of the drug. In embodiments, the ODDF aspect releases the drug immediately and the ERDF aspect thereafter provides continuous release of drug for sustained action.

In embodiments, the immediate release aspect achieves a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, the extended release aspect provides an amount of gaboxadol or pharmaceutically acceptable salt thereof within the patient at about 4 or more hours after administration of the pharmaceutical composition between about 50% to about 100% of the initially administered ODDF dose. In embodiments, the amount of gaboxadol or pharmaceutically acceptable salt thereof within the patient about 4 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 6 or more hours after administration of the pharmaceutical composition between about 50% to about 110% of the initially administered ODDF dose. In embodiments, the amount of gaboxadol or pharmaceutically acceptable salt thereof within the patient about 6 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 8 or more hours after administration of the pharmaceutical composition between about 50% to about 110% of the initially administered ODDF dose. In embodiments, the amount of gaboxadol or pharmaceutically acceptable salt thereof within the patient about 8 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 10 or more hours after administration of the pharmaceutical composition between about 50% to about 110% of the initially administered ODDF dose. In embodiments, the amount of gaboxadol or pharmaceutically acceptable salt thereof within the patient about 10 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the extended release aspect provides an amount of gaboxadol or pharmaceutically acceptable salt thereof within the patient at about 12 or more hours after administration of the pharmaceutical composition between about 50% to about 110% of the initially administered ODDF dose. In embodiments, the amount of gaboxadol or pharmaceutically acceptable salt thereof within the patient about 12 hours after administration of the pharmaceutical composition is more than 50%, 55%, 60%, 65%, 70%, 75%, 80%, 85%, 90%, 95%, 100%, 105%, or 110% of the initially administered ODDF dose.

In embodiments, an ODDF is applied as a coating or band over an ERDF, or as a layer adjacent to an ERDF, to allow direct exposure of the ODDF to the oral cavity and consequent disintegration of the ODDF. In embodiments, the ODDF and the ERDF can be mixed in a chewable resin, e.g., gum. Those skilled in the art are familiar with techniques for applying coatings, bands and layers to fabricate pharmaceutical dosage forms.

Suitable formulations which provide extended release profiles are well-known in the art. For example, coated slow release beads or granules (“beads” and “granules” are used interchangeably herein) in which, e.g., gaboxadol or a pharmaceutically acceptable salt thereof is applied to beads, e.g., confectioners nonpareil beads, and then coated with conventional release retarding materials such as waxes, enteric coatings and the like. In embodiments, beads can be formed in which gaboxadol or pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets. In embodiments, the ODDF is applied as a coating, a layer or a band to a capsule or tablet. In embodiments, slow release cores which are incorporated into tablets or capsules can also provide extended release profiles. For example, gaboxadol or a pharmaceutically acceptable salt thereof can be mixed in a substance or a mixture of substances non-absorbable from the gastrointestinal tract but capable of slow dissolution or loss of drug by leaching, and an outer ODDF layer which is applied to the core by, e.g., compression or spraying. In embodiments, extended release profiles may be provided by multiple layer tablets, each layer having different release properties. Multilayer tableting machines allow incorporation into one tablet of two or more separate layers which may be made to release gaboxadol or a pharmaceutically acceptable salt thereof at different rates. For example, one or more outer layers may be an ODDF, and each other layer an ERDF that exhibits different release rates. In embodiments, gaboxadol or a pharmaceutically acceptable salt thereof is incorporated into porous inert carriers that provide extended release profiles. In embodiments, the porous inert carriers incorporate channels or passages from which the drug diffuses into surrounding fluids. In embodiments, gaboxadol or a pharmaceutically acceptable salt thereof is incorporated into an ion-exchange resin to provide an extended release profile. Prolonged action results from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide an extended release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, absorbable or non-absorbable polymers may be utilized to form ERDFs. Various ERDFs including those discussed above and others that can be utilizable herein are known to those with skill in the art. See, e.g., Fu and Kao, Expert Opin Drug Deliv. 2010 April; 7 (4): 429-444.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as gaboxadol or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, with respect to tablets or capsules, enteric-coated articles are examples of delayed release dosage forms. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

In embodiments, ODDFs with a delayed release formulation aspect are provided that are solid dosage forms containing medicinal substances which disintegrate rapidly, usually within a matter of seconds, when placed upon the tongue, but which also releases a drug (or drugs) at a time other than promptly after administration. Accordingly, in embodiments, modified release dosage forms herein incorporate an ODDF aspect to provide immediate release of a loading dose and then an a delayed release formulation aspect that provides a period in which there is no drug delivery followed by a period of drug delivery to provide drug levels in the blood within a desired therapeutic range for a desirable period of time in excess of the activity resulting from a single dose of the drug. In embodiments, the ODDF aspect releases the drug immediately and then, after a period of delay, a delayed release formulation aspect thereafter provides a single release of drug to provide an additional period of activity. In embodiments, the ODDF aspect releases the drug immediately and then, after a period of delay, a delayed release formulation aspect thereafter provides a continuous release of drug for sustained action.

In embodiments, the immediate release aspect of a ODDF with a delayed release aspect achieves a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, the delayed release aspect provides an amount of gaboxadol or pharmaceutically acceptable salt thereof within the patient at about 1, 2, 3 or 4 or more hours after administration of the pharmaceutical composition between about 50% to about 110% of the initially administered ODDF dose. In embodiments, the amount of gaboxadol or pharmaceutically acceptable salt thereof within the patient about 1, 2, 3 or 4 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 6 or more hours after administration of the pharmaceutical composition between about 50% to about 110% of the initially administered ODDF dose. In embodiments, the amount of gaboxadol or pharmaceutically acceptable salt thereof within the patient about 6 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 8 or more hours after administration of the pharmaceutical composition between about 50% to about 110% of the initially administered ODDF dose. In embodiments, the amount of gaboxadol or pharmaceutically acceptable salt thereof within the patient about 8 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 10 or more hours after administration of the pharmaceutical composition between about 50% to about 110% of the initially administered ODDF dose. In embodiments, the amount of gaboxadol or pharmaceutically acceptable salt thereof within the patient about 10 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the delayed release formulation aspect provides an amount of gaboxadol or pharmaceutically acceptable salt thereof within the patient at about 12 or more hours after administration of the pharmaceutical composition between about 50% to about 110% of the initially administered ODDF dose. In embodiments, the amount of gaboxadol or pharmaceutically acceptable salt thereof within the patient about 12 hours after administration of the pharmaceutical composition is more than 50%, 55%, 60%, 65%, 70%, 75%, 80%, 85%, 90%, 95%, 100%, 105%, or 110% of the initially administered ODDF dose.

Delayed release dosage forms are known to those skilled in the art. For example, coated delayed release beads or granules (“beads” and “granules” are used interchangeably herein) in which, e.g., gaboxadol or a pharmaceutically acceptable salt thereof is applied to beads, e.g., confectioners nonpareil beads, and then coated with conventional release delaying materials such as waxes, enteric coatings and the like. In embodiments, beads can be formed in which gaboxadol or pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of gaboxadol or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets. In embodiments, the ODDF is applied as a coating, a layer or a band to the capsule or tablet. In embodiments, delayed release cores which are incorporated into tablets or capsules can also provide delayed release profiles. For example, gaboxadol or a pharmaceutically acceptable salt thereof can be mixed in a substance or a mixture of substances non-absorbable from the gastrointestinal tract but capable of slow dissolution or loss of drug by leaching, and an outer ODDF layer which is applied to the core by, e.g., compression or spraying. In embodiments, delayed release profiles may be provided by multiple layer tablets, each layer having different release properties. Multilayer tableting machines allow incorporation into one tablet of two or more separate layers which may be made to release gaboxadol or a pharmaceutically acceptable salt thereof at different rates after a period of delay. For example, one or more outer layers may be an ODDF, and each other layer a delayed release dosage form that exhibits different release rates. In embodiments, gaboxadol or a pharmaceutically acceptable salt thereof is incorporated into porous inert carriers that provide delayed release profiles. In embodiments, the porous inert carriers incorporate channels or passages from which the drug diffuses into surrounding fluids. In embodiments, gaboxadol or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, an ODDF is applied as a coating or band over a delayed release dosage form, or as a layer adjacent to a delayed release dosage form, to allow direct exposure of the ODDF to the oral cavity and consequent disintegration of the ODDF. In embodiments, the ODDF and a delayed release dosage form can be mixed in a chewable resin, e.g., gum. Those skilled in the art are familiar with techniques for applying coatings, bands and layers to fabricate pharmaceutical dosage forms.

In embodiments, modified release pharmaceutical compositions herein include pulsatile release dosage formulations (PRDFs). Pulsatile drug release involves rapid release of defined or discrete amounts of a drug (or drugs) such as gaboxadol or a pharmaceutically acceptable salt thereof after a lag time following an initial release of drug. In embodiments, PRDFs can provide a single pulse. In embodiments, PRDFs can provide multiple pulses over time. Various PRDFs are known to those with skill in the art.

In embodiments, a PRDF can be a capsule. In embodiments, release after a lag time is provided by a system that uses osmotic pressure to cause release of a plug. In this system, gaboxadol or a pharmaceutically acceptable salt thereof is contained in an insoluble capsule shell sealed by an osmotically responsive plug, e.g., a hydrogel, which is pushed away by swelling or erosion. When the seal is broken the drug is released as a pulse from the capsule body. Contact with gastrointestinal fluid or dissolution medium causes the plug to swell, either pushing itself out of the capsule or causing the capsule to rupture after the lag-time. Position & dimensions of the plug can control lag-time. For rapid release of drug effervescent or disintegrating agents may be added. Effervescent materials can cause an increase in pressure thus aiding or causing expulsion of the plug. Examples of suitable plug material may be swellable materials coated with permeable polymer (polymethacrylates), erodible compressed polymer (HPMC, polyvinyl alcohol), congealed melted polymer (glyceryl monooleate), and enzymatically controlled erodible polymers such as pectin. In embodiments, an insoluble capsule contains multiple drug compartments separated by osmotically activated plugs. When a first plug is exposed to the environmental fluids, the first compartment opens, drug is released and the adjacent plug is exposed. The process continues until no sealed compartment are left. Lag time between pulses can be further controlled by varying the thickness of the plug and the properties of the materials from which the plug is made. More hygroscopic materials will absorb fluid faster and will swell faster. In embodiments, a membrane may be substituted for the plug. If effervescent materials are included in one or more compartments, fluids pass through the membrane by osmosis and the effervescent action and pressure increase causes the membrane to rupture, thereby releasing the drug. In embodiments, the membrane(s) are erodible and dissolve to release the contents of the compartment(s). Varying the thickness, porosity and properties of materials of the membrane can allow further control of lag time between pulses. In embodiments, a PRDF can be a tablet. In embodiments, single pulse tablets involve a core containing gaboxadol or a pharmaceutically acceptable salt thereof surrounded by one or more layers of swellable, rupturable coatings. In embodiments, a rupturable coating surrounds a swellable layer. As the swellable layer expands, it causes the rupturable coating to rupture, thereby releasing the drug from the core. Swellable materials such as hydrogels are well known. In embodiments, an inner swelling layer can contain a superdisintegrant, e.g., croscarmellose sodium, and an outer rupturable layer can be made of a polymeric porous materials such as polyethylene oxides, ethylcellulose and the like. Porous film coats of sucrose may also be suitable. In embodiments, multiple pulse tablets incorporate multiple layers surrounding a core. As a first outermost layer erodes and releases the drug contained within the layer, an underlying layer is exposed, thus releasing drug after a predetermined lag time. The process repeats until the innermost core is exposed.

In embodiments, PRDFs can incorporate ODDFs that are solid dosage forms containing medicinal substances which disintegrate rapidly, usually within a matter of seconds, when placed upon the tongue, but which also releases a drug (or drugs) in pulsatile fashion. Accordingly, in embodiments, modified release dosage forms herein incorporate an ODDF aspect to provide immediate release of a loading dose and a PRDF aspect that provides a period in which there is no drug delivery (lag time) followed by pulsatile drug delivery to provide drug levels in the blood within a desired therapeutic range for a desirable period of time in excess of the activity resulting from a single dose of the drug. In embodiments, the ODDF aspect releases the drug immediately and then, after a period of delay, the PRDF aspect thereafter provides a single pulse release of drug to provide an additional period of activity. In embodiments, the ODDF aspect releases the drug immediately and then, after a period of delay, the PRFD aspect thereafter provides multiple pulsatile release of drug for prolonged therapeutic effect.

In embodiments, the immediate release aspect of a ODDF with a PRDF aspect achieves a Tₘₐₓ of 20 minutes or less, a Tₘₐₓ of 19 minutes or less, a Tₘₐₓ of 18 minutes or less, a Tₘₐₓ of 17 minutes or less, a Tₘₐₓ of 16 minutes or less, a Tₘₐₓ of 15 minutes or less, a Tₘₐₓ of 14 minutes or less, a Tₘₐₓ of 13 minutes or less, a Tₘₐₓ of 12 minutes or less, a Tₘₐₓ of 11 minutes or less, a Tₘₐₓ of 10 minutes or less, a Tₘₐₓ of 9 minutes or less, a Tₘₐₓ of 8 minutes or less, a Tₘₐₓ of 7 minutes or less, a Tₘₐₓ of 6 minutes or less, or a Tₘₐₓ of 5 minutes or less. In embodiments, a PRDF aspect provides an amount of gaboxadol or pharmaceutically acceptable salt thereof within the patient at about 0.5, 1, 2, 3 or 4 or more hours after administration of the pharmaceutical composition between about 50% to about 110% of the initially administered ODDF dose. In embodiments, the amount of gaboxadol or pharmaceutically acceptable salt thereof within the patient about 0.5, 1, 2, 3 or 4 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 6 or more hours after administration of the pharmaceutical composition between about 50% to about 110% of the initially administered ODDF dose. In embodiments, the amount of gaboxadol or pharmaceutically acceptable salt thereof within the patient about 6 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 8 or more hours after administration of the pharmaceutical composition between about 50% to about 110% of the initially administered ODDF dose. In embodiments, the amount of gaboxadol or pharmaceutically acceptable salt thereof within the patient about 8 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 10 or more hours after administration of the pharmaceutical composition between about 50% to about 110% of the initially administered ODDF dose. In embodiments, the amount of gaboxadol or pharmaceutically acceptable salt thereof within the patient about 10 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, a PRDF aspect provides an amount of gaboxadol or pharmaceutically acceptable salt thereof within the patient at about 12 or more hours after administration of the pharmaceutical composition between about 50% to about 110% of the initially administered ODDF dose. In embodiments, the amount of gaboxadol or pharmaceutically acceptable salt thereof within the patient about 12 hours after administration of the pharmaceutical composition is more than 50%, 55%, 60%, 65%, 70%, 75%, 80%, 85%, 90%, 95%, 100%, 105%, or 110% of the initially administered ODDF dose. In embodiments, the PRDF delivers one pulse in accordance with the above amounts. In embodiments, the PRDF delivers two pulses in accordance with the above amounts. In embodiments, the PRDF delivers three pulses in accordance with the above amounts. In embodiments, the PRDF delivers four pulses in accordance with the above amounts. In embodiments, the PRDF delivers five pulses in accordance with the above amounts. In embodiments, the PRDF delivers six pulses in accordance with the above amounts. In embodiments, the PRDF delivers seven pulses in accordance with the above amounts. In embodiments, the PRDF delivers eight pulses in accordance with the above amounts. In embodiments, the PRDF delivers nine pulses in accordance with the above amounts. The pulses may be provided in intervals separated by 0.25 h, 0.5 h, 0.75 h, 1 h, 1.25 h, 1.5 h, 1.75 h, 2, h, 2.25 h, 2.5 h, 2.75 h, 3 h, 3.25 h, 3.5 h, 3.75 h, 4 h, 4.25 h, 4.5 h, 4.75 h, 5 h, 5.5 h, 6 h, 7 h, 8 h, 9 h, 10 h, 11 h, or 12 h. In embodiments the amount of gaboxadol or a pharmaceutically acceptable salt thereof released with each pulse may vary.

In embodiments, an ODDF is applied as a coating or band over a PRDF, or as a layer adjacent to a PRDF, to allow direct exposure of the ODDF to the oral cavity and consequent disintegration of the ODDF. In embodiments, the ODDF and a PRDF can be mixed in a chewable resin, e.g., gum. Those skilled in the art are familiar with techniques for applying coatings, bands and layers to fabricate pharmaceutical dosage forms.

Embodiments described herein provide that a patient with Angelman syndrome, Fragile X syndrome, or Fragile X-associated tremor/ataxia syndrome and in need thereof is administered a modified release pharmaceutical composition including gaboxadol or a pharmaceutically acceptable salt thereof. Gaboxadol or pharmaceutically acceptable salt thereof may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic or theophylline acetic acid addition salts, as well as the 8-halotheophyllines, for example 8-bromo-theophylline. In other suitable embodiments, inorganic acid addition salts, including but not limited to, hydrochloric, hydrobromic, sulfuric, sulfamic, phosphoric or nitric acid addition salts may be used.

In embodiments, gaboxadol is provided as gaboxadol monohydrate. One skilled in the art will readily understand that the amounts of active ingredient in a pharmaceutical composition will depend on the form of gaboxadol provided. For example, pharmaceutical compositions including 5.0, 10.0, or 15.0 mg gaboxadol correspond to 5.6, 11.3, or 16.9 mg gaboxadol monohydrate.

In embodiments, gaboxadol is crystalline, such as the crystalline hydrochloric acid salt, the crystalline hydrobromic acid salt, or the crystalline zwitter ion monohydrate. In embodiments, gaboxadol is provided as a crystalline monohydrate.

Deuteration of pharmaceuticals to improve pharmacokinetics (PK), pharmacodynamics (PD), and toxicity profiles, has been demonstrated previously with some classes of drugs. Accordingly the use of deuterium enriched gaboxadol is contemplated and within the scope of the methods and compositions described herein. Deuterium can be incorporated in any position in replace of hydrogen synthetically, according to the synthetic procedures known in the art. For example, deuterium may be incorporated to various positions having an exchangeable proton, such as the amine N—H, via proton-deuterium equilibrium exchange. Thus, deuterium may be incorporated selectively or non-selectively through methods known in the art to provide deuterium enriched gaboxadol. See Journal of Labeled Compounds and Radiopharmaceuticals 19 (5) 689-702 (1982).

Deuterium enriched gaboxadol may be described by the percentage of incorporation of deuterium at a given position in the molecule in the place of hydrogen. For example, deuterium enrichment of 1% at a given position means that 1% of molecules in a given sample contain deuterium at that specified position. The deuterium enrichment can be determined using conventional analytical methods, such as mass spectrometry and nuclear magnetic resonance spectroscopy. In some embodiments deuterium enriched gaboxadol means that the specified position is enriched with deuterium above the naturally occurring distribution (i.e., above about.0156%). In embodiments deuterium enrichment is no less than about 1%, no less than about 5%, no less than about 10%, no less than about 20%, no less than about 50%, no less than about 70%, no less than about 80%, no less than about 90%, or no less than about 98% of deuterium at a specified position.

In embodiments methods of treating a patient with Angelman syndrome, Fragile X syndrome, or Fragile X-associated tremor/ataxia syndrome include administering to a patient in need thereof a modified release pharmaceutical composition including about 0.05 mg to about 100 mg gaboxadol or a pharmaceutically acceptable salt thereof.

In embodiments, the modified release pharmaceutical compositions include 0.1 mg to 75 mg, 0.1 mg to 70 mg, 0.1 mg to 65 mg, 0.1 mg to 55 mg, 0.1 mg to 50 mg, 0.1 mg to 45 mg, 0.1 mg to 40 mg, 0.1 mg to 35 mg, 0.1 mg to 30 mg, 0.1 mg to 25 mg, 0.1 mg to 20 mg, 0.1 mg to 15 mg, 0.1 mg to 10 mg, 0.5 mg to 75 mg, 0.5 mg to 70 mg, 0.5 mg to 65 mg, 0.5 mg to 55 mg, 0.5 mg to 50 mg, 0.5 mg to 45 mg, 0.5 mg to 40 mg, 0.5 mg to 35 mg, 0.5 mg to 30 mg, 0.5 mg to 25 mg, 0.5 mg to 20 mg, 0.5 to 15 mg, 0.5 to 10 mg, 1 mg to 75 mg, 1 mg to 70 mg, 1 mg to 65 mg, 1 mg to 55 mg, 1 mg to 50 mg, 1 mg to 45 mg, 1 mg to 40 mg, 1 mg to 35 mg, 1 mg to 30 mg, 1 mg to 25 mg, 1 mg to 20 mg, 1 mg to 15 mg, 1 mg to 10 mg, 1.5 mg to 75 mg, 1.5 mg to 70 mg, 1.5 mg to 65 mg, 1.5 mg to 55 mg, 1.5 mg to 50 mg, 1.5 mg to 45 mg, 1.5 mg to 40 mg, 1.5 mg to 35 mg, 1.5 mg to 30 mg, 1.5 mg to 25 mg, 1.5 mg to 20 mg, 1.5 mg to 15 mg, 1.5 mg to 10 mg, 2 mg to 75 mg, 2 mg to 70 mg, 2 mg to 65 mg, 2 mg to 55 mg, 2 mg to 50 mg, 2 mg to 45 mg, 2 mg to 40 mg, 2 mg to 35 mg, 2 mg to 30 mg, 2 mg to 25 mg, 2 mg to 20 mg, 2 mg to 15 mg, 2 mg to 10 mg, 2.5 mg to 75 mg, 2.5 mg to 70 mg, 2.5 mg to 65 mg, 2.5 mg to 55 mg, 2.5 mg to 50 mg, 2.5 mg to 45 mg, 2.5 mg to 40 mg, 2.5 mg to 35 mg, 2.5 mg to 30 mg, 2.5 mg to 25 mg, 2.5 mg to 20 mg, 2.5 mg to 15 mg, 2.5 mg to 10 mg, 3 mg to 75 mg, 3 mg to 70 mg, 3 mg to 65 mg, 3 mg to 55 mg, 3 mg to 50 mg, 3 mg to 45 mg, 3 mg to 40 mg, 3 mg to 35 mg, 3 mg to 30 mg, 3 mg to 25 mg, 3 mg to 20 mg, 3 mg to 15 mg, 3 mg to 10 mg, 3.5 mg to 75 mg, 3.5 mg to 70 mg, 3.5 mg to 65 mg, 3.5 mg to 55 mg, 3.5 mg to 50 mg, 3.5 mg to 45 mg, 3.5 mg to 40 mg, 3.5 mg to 35 mg, 3.5 mg to 30 mg, 3.5 mg to 25 mg, 3.5 mg to 20 mg, 3.5 mg to 15 mg, 3.5 mg to 10 mg, 4 mg to 75 mg, 4 mg to 70 mg, 4 mg to 65 mg, 4 mg to 55 mg, 4 mg to 50 mg, 4 mg to 45 mg, 4 mg to 40 mg, 4 mg to 35 mg, 4 mg to 30 mg, 4 mg to 25 mg, 4 mg to 20 mg, 4 mg to 15 mg, 4 mg to 10 mg, 4.5 mg to 75 mg, 4.5 mg to 70 mg, 4.5 mg to 65 mg, 4.5 mg to 55 mg, 4.5 mg to 50 mg, 4.5 mg to 45 mg, 4.5 mg to 40 mg, 4.5 mg to 35 mg, 4.5 mg to 30 mg, 4.5 mg to 25 mg, 4.5 mg to 20 mg, 4.5 mg to 15 mg, 4.5 mg to 10 mg, 5 mg to 75 mg, 5 mg to 70 mg, 5 mg to 65 mg, 5 mg to 55 mg, 5 mg to 50 mg, 5 mg to 45 mg, 5 mg to 40 mg, 5 mg to 35 mg, 5 mg to 30 mg, 5 mg to 25 mg, 5 mg to 20 mg, 5 mg to 15 mg, or 5 mg to 10 mg, gaboxadol or a pharmaceutically acceptable salt thereof.

In embodiments, pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg gaboxadol or a pharmaceutically acceptable salt thereof.

In embodiments, pharmaceutical compositions include 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, or 20 mg gaboxadol or a pharmaceutically acceptable salt thereof or amounts that are multiples of such doses. In embodiments, pharmaceutical compositions include 2.5 mg, 5 mg, 7.5 mg, 10 mg, 15 mg, or 20 mg gaboxadol or a pharmaceutically acceptable salt thereof.

In embodiments, ODDFs include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 7 mg, 7.5 mg, 10 mg, 12.5 mg, 15 mg, 17.5 mg, or 20 mg gaboxadol or a pharmaceutically acceptable salt thereof or amounts that are multiples of such doses.

In embodiments, ERDFs include from about 1 mg to about 100 mg gaboxadol or a pharmaceutically acceptable salt thereof In embodiments, ERDFs include 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, delayed release dosage forms include from about 0.05 mg to about 100 mg gaboxadol or a pharmaceutically acceptable salt thereof In embodiments, delayed release dosage forms include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, PRDFs include one or more pulse providing domains having from about 0.05 mg to about 100 mg gaboxadol or a pharmaceutically acceptable salt thereof In embodiments, PRDFs include 0.05 mg, 0.1 mg, 0.25 mg, 0.5 mg, 0.75 mg, 1 mg, 1.25 mg, 1.5 mg, 1.75 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100mg gaboxadol or a pharmaceutically acceptable salt thereof.

In embodiments, a modified release pharmaceutical composition provides an in vivo plasma profile having Cₘₐₓ less than about 2500 ng/ml, 2000 ng/ml, 1750 ng/ml, 1500 ng/ml, 1250 ng/ml, 1000 ng/ml, 750 ng/ml, 500 ng/ml, 450 ng/ml, 400 ng/ml, 350 ng/ml, 300 ng/ml, 250 ng/ml, 200 ng/ml, 150 ng/ml, 100 ng/ml, 50 ng/ml or 25 ng/ml. In embodiments, ODDFs herein provide an in vivo plasma profile having a AUC_{0-∞} of less than about, e.g., 900 ng·hr/ml, 850 ng·hr/ml, 800 ng·hr/ml, 750 ng·hr/ml, or 700 ng·hr/ml 650 ng·hr/ml, 600 ng·hr/ml, 550 ng·hr/ml, 500 ng·hr/ml, or 450 ng·hr/ml. In embodiments, ODDFs herein provide an in vivo plasma profile having a AUC_{0-∞} of less than about, e.g., 400 ng·hr/ml, 350 ng·hr/ml, 300 ng·hr/ml, 250 ng·hr/ml, or 200 ng·hr/ml. In embodiments, ODDFs herein provide an in vivo plasma profile having a AUC_{0-∞} of less than about, e.g., 150 ng·hr/ml, 100 ng·hr/ml, 75 ng·hr/ml, or 50 ng·hr/ml.

In embodiments, modified release pharmaceutical compositions with different drug release profiles may be combined to create a two phase or three-phase release profile. For example, as mentioned above, pharmaceutical compositions may be provided with an immediate release and an extended release profile. In embodiments, modified release pharmaceutical compositions may be provided with an immediate release, extended release and delayed release profile. Compositions may be prepared using a pharmaceutically acceptable “carrier” composed of excipients that are considered safe and effective. The “carrier” includes all components present in the pharmaceutical formulation other than the active ingredient or ingredients. The term “carrier” includes, but is not limited to, excipients such as diluents, binders, lubricants, disintegrants, fillers, and coating compositions.

In embodiments, pharmaceutical compositions described herein are administered once, twice, three times daily, four times daily, or every other day. In embodiments, a pharmaceutical composition described herein is provided to the patient in the evening or in the morning. In embodiments, a pharmaceutical composition described herein is provided to the patient once in the evening and once in the morning. In embodiments, the total amount of gaboxadol or a pharmaceutically acceptable salt thereof administered to a subject in a 24-hour period is 1 mg to 100 mg. In embodiments, the total amount of gaboxadol or a pharmaceutically acceptable salt thereof administered to a subject in a 24-hour period is 1 mg to 50 mg. In embodiments, the total amount of gaboxadol or a pharmaceutically acceptable salt thereof administered to a subject in a 24-hour period is 1 mg to 25 mg. In embodiments, the total amount of gaboxadol or a pharmaceutically acceptable salt thereof administered to a subject in a 24-hour period is 1 mg to 20 mg. In embodiments, the total amount of gaboxadol or a pharmaceutically acceptable salt thereof administered to a subject in a 24-hour period is 5 mg, 10 mg, or 15 mg. In embodiments, the total amount of gaboxadol or a pharmaceutically acceptable salt thereof administered to a subject in a 24-hour period is 20 mg.

In embodiments, provided herein are methods of treating Angelman syndrome, Fragile X syndrome or Fragile X-associated tremor/ataxia syndrome including administering to a patient in need thereof a modified release pharmaceutical composition including gaboxadol or a pharmaceutically acceptable salt thereof wherein the composition provides improvement in at least one symptom of Angelman syndrome, Fragile X syndrome or Fragile X-associated tremor/ataxia syndrome. Symptoms may include, but are not limited to, tremors such as intention tremor, resting tremor, rigidity, ataxia, bradykinesia, gait, speech impairment, vocalization difficulties, cognition impairment, motor activity deficits, clinical seizure, hypotonia, hypertonia, feeding difficulty, drooling, mouthing behavior, sleep difficulties, hand flapping, easily provoked laughter, short attention span, reduced sensation, numbness or tingling, pain, muscle weakness in the lower limbs, inability to control the bladder or bowel, chronic pain syndromes, such as fibromyalgia and chronic migraine, hypothyroidism, hypertension, sleep apnea, vertigo, olfactory dysfunction, and hearing loss, short-term memory loss, loss of executive function, impulse control, self-monitoring, focusing attention appropriately, cognitive flexibility psychiatric symptoms such as anxiety, depression, moodiness, or irritability. In embodiments, provided herein are improvements in cognition. Cognition refers to the mental processes involved in gaining knowledge and comprehension, such as thinking, knowing, remembering, judging, and problem solving. These higher-level functions of the brain encompass language, imagination, perception, and the planning and execution of complex behaviors.

In embodiments, provided herein are methods of treating Angelman syndrome, Fragile X syndrome or Fragile X-associated tremor/ataxia syndrome including administering to a patient in need thereof a pharmaceutical composition including gaboxadol or a pharmaceutically acceptable salt thereof wherein the composition provides improvement of at least one symptom for more than 4 hours after administration of the pharmaceutical composition to the patient. In embodiments, provided herein is improvement of at least one symptom for more than 6 hours after administration of the pharmaceutical composition to the patient. In embodiments, provided herein is improvement of at least one symptom for more than, e.g., 8 hours, 10 hours, 12 hours, 15 hours, 18 hours, 20 hours, or 24 hours after administration of the pharmaceutical composition to the patient. In embodiments, provided herein is improvement in at least one symptom for at least, e.g., 8 hours, 10 hours, 12 hours, 15 hours, 18 hours, 20 hours, or 24 hours after administration of the pharmaceutical composition to the patient. In embodiments, provided herein is improvement in at least one symptom for 12 hours after administration of the pharmaceutical composition to the patient.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value.

“Improvement” refers to the treatment of a subject having Angelman syndrome, Fragile X syndrome or Fragile X-associated tremor/ataxia syndrome measured relative to at least one symptom.

“PK” refers to the pharmacokinetic profile. Cₘₐₓ is defined as the highest plasma drug concentration estimated during an experiment (ng/ml) following administration of a drug. Tₘₐₓ is defined as the time when Cₘₐₓ is estimated (min). AUC_{0-∞} is the total area under the plasma drug concentration-time curve, from drug administration until the drug is eliminated (ng·hr/ml). The area under the curve is governed by clearance. Clearance is defined as the volume of blood or plasma that is totally cleared of its content of drug per unit time (ml/min).

“Treating” or “treatment” refers to alleviating or delaying the appearance of clinical symptoms of a disease or condition in a subject that may be afflicted with or predisposed to the disease or condition, but does not yet experience or display clinical or subclinical symptoms of the disease or condition. In certain embodiments, “treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” also refers to inhibiting the disease or condition, e.g., arresting or reducing its development or at least one clinical or subclinical symptom thereof “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic (curative) treatment are two separate embodiments of the disclosure herein.

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount” means a dosage sufficient to alleviate one or more symptom of a disorder, disease, or condition being treated, or to otherwise provide a desired pharmacological and/or physiologic effect.

“Patient in need thereof” may include individuals that have been diagnosed with Angelman syndrome, Fragile X syndrome or Fragile X-associated tremor/ataxia syndrome. The methods may be provided to any individual including, e.g., wherein the patient is a neonate, infant, a pediatric patient (6 months to 12 years), an adolescent patient (age 12-18 years) or an adult (over 18 years).

The following examples are included to help illustrate and/or augment the description herein. The examples are not to be construed as limiting the disclosure herein in any way.

### EXAMPLE 1

Gaboxadol 15 mg Orally Disintegrating Tablet

| | | | |
|---|---|---|---|
| | Compendial | | Unit Strength |
| Components | Testing | Function | 15 mg |

Intragranular

| | | | |
|---|---|---|---|
| Gaboxadol^{†} | — | Active | mg/tablet |
| (equivalent anhydrous) | | | 16.94 |
| Aspartame | NF | Sweetener | 2.00 |
| Peppermint (Natural | — | Flavor | 1.00 |
| and Artificial) | | | |
| Monoammonium | — | Sweetener | 1.00 |
| Glycyrrhizinate | | | |
| Lactose Monohydrate | NF | Diluent | 63.87 |
| (modifiedspray-dried) | | | |
| Crospovidone | NF | Disintegrant | 10.00 |
| Mannitol | USP | Diluent | 104.00 |
| FD&C Blue No. 2 | — | Colorant | 0.20 |
| Aluminum Lake | | | |
| Magnesium Stearate | NF | Lubricant | 1.00 |
| (non-bovine) | | | |
| Total Tablet Weight | | | 200.0 |

^{†}Conversion Factor: 1.129 mg of monohydrate = 1.0 mg of anhydrous

The gaboxadol ODT formulation is prepared by blending the active drug, aspartame, peppermint flavor, monoammonium glycyrrhizinate, lactose monohydrate, crospovidone, mannitol and FD&C blue #2 in a suitable diffusional blender until uniform. The magnesium stearate is added and the material is blended. The final lubricated blend is compressed on a tablet press.

### EXAMPLE 2

### Gaboxadol Orally Disintegrating Film

A hydrophilic film-forming agent is made from a graft copolymer having a film-forming block of polyvinyl alcohol (PVA) Kollicoat IR® (marketed by BASF), molecular weight about 45,000 Da, and a polyethylene glycol (PEG) plasticizer. The gelling agent is Gelcarin 379® (commercially available from FMC Biopolymer), a compound of the carrageenan family. Kollicoat IR® is introduced into 70% of the amount of purified water under stirring. Agitation is maintained until dissolution of Kollicoat IR®. Since gas bubbles are generated, the solution may be dissolved under a vacuum or the solution can stand (its viscosity is very low) until the gas is dispersed. Tween 80 is incorporated to the stirred solution and flavorings (condensed licorice extract and essential oil of peppermint) and sweetener (acesulfame potassium) are added. Stirring is continued until complete dissolution of all powder. Gaboxadol is introduced with stirring until it is dispersed in the mixture, then the remaining water (30%) is added. Gelcarin 379® is incorporated into suspension under agitation to prevent the formation of aggregates. The final mixture consists of gaboxadol 6% w/w, Kollicoat IR® 15% w/w, Gelcarin 379® 5% w/w, Tween 80 0.2% w/w, acesulfame potassium 0.05% w/w, flavorings 1.5% w/w, purified water qs. Mixing aliquots are then coated on a polyester backing and dried in a type Lab Dryer Coater (Mathis equipment). The coated surfaces are cut using a manual press in 6 cm² units, and then manually packaged in sealed bags.

### EXAMPLE 3

### Plasma Concentration Profiles of Gaboxadol Single Dose

### Conventional Capsule Formulations

The following Example provides the plasma concentration profiles and dose proportionality of gaboxadol monohydrate following single oral doses ranging from 2.5 to 20 mg. The absolute bioavailability of gaboxadol monohydrate capsules ranging from 2.5 to 20 mg is also assessed.

This study was composed of separate groups of 10 healthy adult subjects (at least 4 of each gender) who participated in a 6-period, double-blind, randomized, crossover study designed to access the dose proportionality and absolute bioavailability of 5 single oral doses of gaboxadol across the dose range of 2.5 to 20 mg. The order in which the subjects received the 5 single oral doses of gaboxadol (2.5; 5; 10; 15; and 20 mg) was randomized within Treatment Periods 1 through 5. Each subject was expected to complete all 6 treatment periods and there was a washout of at least 4 days between each treatment period.

Each oral dosing within Treatment Periods consisted of 2 capsules of test drug taken simultaneously at each scheduled dosing. The treatment designations for the orally administered study drugs were as follows: Treatment A—one 2.5 mg gaboxadol capsule and 1 matching placebo capsule; Treatment B—one 5 mg gaboxadol capsule and 1 matching placebo capsule; Treatment C—one 10 mg gaboxadol capsule and 1 matching placebo capsule; Treatment D—one 15 mg gaboxadol capsule and 1 matching placebo capsule; and Treatment E—20 mg gaboxadol (two 10 mg gaboxadol capsules). Subjects received their study drug after an overnight fast with 240 mL of water in the morning about 8:00 AM. Water was permitted ad libitum except within 1 hour prior to and after study drug administration. No food was allowed for 4 hours post dose.

For each subject in each treatment, plasma and urine samples were collected over 16 hours post-dosing for the determination of pharmacokinetic parameters (e.g., AUC, Cₘₐₓ, Tₘₐₓ, apparent t_{1/2}, cumulative urinary excretion, renal clearance, clearance, and steady-state volume of distribution, as appropriate). AUC and Cₘₐₓ for gaboxadol were potency adjusted to facilitate comparison of pharmacokinetic data across studies. TABLE 1 provides the individual potency-adjusted pharmacokinetic parameters of gaboxadol following single oral doses (2.5, 5, 10, 15, and 20 mg).

TABLE I
Pharmacokinetic parameters for gaboxadol following oral and
IV administration

| | Geometric Mean (N = 10) | | | | | | Slope |
|---|---|---|---|---|---|---|---|
| | | | 10 mg | 10 mg | | | (90% |
| Parameter | 2.5 mg | 5 mg | Oral | I.V. | 15 mg | 20 mg | CI)^{††} |
| AUC_{0-∞} | 90 | 171 | 346 | 380 | 539 | 669 | 0.98 (0.95, |
| (ng · hr/mL) | | | | | | | 1.01) |
| Cₘₐₓ (ng/mL)^{†} | 61 | 110 | 232 | 212 | 382 | 393 | 0.95 (0.88, |
| | | | | | | | 1.02) |
| Tₘₐₓ (hr)^{‡} | 0.5 | 0.6 | 0.5 | — | 0.5 | 0.6 | |
| Apparent t_{b} (hr)^{f} | 1.5 | 1.5 | 1.6 | 1.5 | 1.5 | 1.6 | |
| CL/F (mL/min)^{g} | 461 | 488 | 476 | 438 | 469 | 499 | |
| F_{c} (%) | 43 | 45 | 53 | 53 | 50 | 53 | |
| CL_{R} (mL/min) | 196 | 222 | 250 | 208 | 234 | 265 | |

| | |
|---|---|
| F (%) (90% CI)^{#} | 92% (0.86, 0.97) |

^{†}C_{col} (ng/mL) for 10 mg IV.
^{‡}Median.
^{f}Harmonic mean.
^{g}CL (mL/min) for 10 mg IV.
^{#}Bioavailability relative to 10 mg I.V. reference based on pooled dose-adjusted (to 10 mg) oral AUC_{0-∞} values.
^{††}Dose proportionality assessment of oral treatments only.

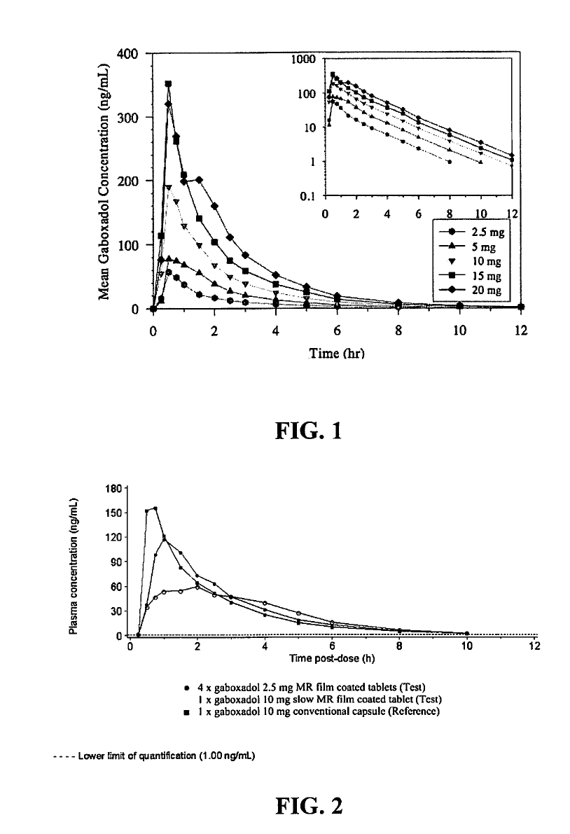

FIG. 1 shows the arithmetic mean plasma concentration-time profiles of gaboxadol following single oral doses (2.5, 5, 10, 15, and 20 mg). The bioavailability of gaboxadol is approximately 92%. Plasma AUC_{0-∞} and Cₘₐₓ of gaboxadol show dose proportional increases and appear to be linear over the entire dose range examined, from of 2.5 to 20 mg. The time to peak plasma concentrations (Tₘₐₓ 30-60 min) and the half-life (t^{1/2} of 1.5 h) for gaboxadol appear to be independent of dose across the gaboxadol dose range of 2.5 to 20 mg. The excretion of gaboxadol is mainly via urine, where 96.5% of the dose is recovered; 75% is recovered within 4 hours after administration.

### EXAMPLE 4

### Pharmacokinetics of Modified Release Formulations of Gaboxadol

With the aim of decreasing the peak plasma concentrations of gaboxadol in order to increase the therapeutic window of the drug, two different modified release formulations were developed. The modified release (MR) tablets are matrix formulations based on different content of the water insoluble hydroxyl propyl methylcellulose (HPMC) controlling the dissolution of gaboxadol. The two different (fast and slow) matrix formulations were both developed in the strengths of 2.5 mg, 5.0 mg, 7.5 mg and 10 mg gaboxadol. A relative bioavailability study has been conducted to compare the PK of the matrix formulations to the PK of the conventional capsule of gaboxadol. The 2.5 mg fast and the 10 mg slow matrix formulations were tested in the relative bioavailability study. Plasma concentration-time profiles for a single administration of four gaboxadol 2.5 mg fast modified release film coated tablets, a single gaboxadol 10 mg slow modified release film coated tablet and a single gaboxadol 10 mg conventional capsule are shown in FIG. 2. The results of statistical analyses performed on the pharmacokinetic parameters of gaboxadol are summarized in TABLE II.

TABLE II
Statistical Analyses of the Pharmacokinetic Parameters of Gaboxadol
Following Single Dose Administrations of 10 mg Gaboxadol
Estimated Differences or Ratios (95% CI)

| | 4 × Gaboxadol 2.5 mg MR | | 1 × Gaboxadol 10 mg Slow MR | |
|---|---|---|---|---|
| | Film Coated Tablets vs. | | Film Coated Tablet vs. | |
| | 1 × Gaboxadol 10 mg | | 1 × Gaboxadol 10 mg | |
| | Conventional Capsule | | Conventional Capsule | |
| Parameter | (n = 18) | | (n = 18) | |
| AUC_{0-inf} | 0.959 | (0.899, 1.02) | 0.810 | (0.760, 0.864) |
| (h · | | | | |
| ng/mL) | | | | |
| Cₘₐₓ | 0.777 | (0.687, 0.880) | 0.375 | (0.331, 0.424) |
| (ng/mL) | | | | |
| AUC₀₋ₜ | 0.957 | (0.897, 1.02) | 0.804 | (0.754, 0.858) |
| (h · | | | | |
| ng/mL) | | | | |
| t_{1/2} | 0.0245 | (−0.0250, 0.0739) | −0.00593 | (−0.0554, 0.0435) |
| (h) | | | | |

Estimated ratios are presented for AUC_{0-inf}, Cₘₐₓ, and AUC₀₋ₜ.
Estimated differences are presented for t_{1/2}.
n = number of subjects

The plasma pharmacokinetic parameters of gaboxadol are shown in TABLE III.

TABLE III
Summary of Pharmacokinetic Parameters of Gaboxadol Following
Single Dose Administrations of 10 mg Gaboxadol

| | 4 × Gaboxadol | | 1 × Gaboxadol | | 1 × Gaboxadol | |
|---|---|---|---|---|---|---|
| | 2.5 mg MR Film | | 10 mg Slow MR Film | | 10 mg Conven- | |
| | Coated Tablets | | Coated Tablet | | tional Capsule | |
| Parameter | (n = 18) | | (n = 18) | | (n = 18) | |
| AUC_{0-inf} | 325 | (15.2) | 274 | (16.2) | 338 | (16.4) |
| (h · | | | | | | |
| ng/mL) | | | | | | |
| AUC₀₋ₜ | 322 | (15.2) | 270 | (16.4) | 335 | (16.4) |
| (h · | | | | | | |
| ng/mL) | | | | | | |
| Cₘₐₓ | 151 | (26.3) | 67.5 | (15.2) | 201 | (35.7) |
| (ng/mL) | | | | | | |
| tₘₐₓ (h) | 1.0 | (0.5, 2.5) | 1.5 | (0.75, 3.0) | 0.583 | (0.5, 2.0) |
| t_{1/2} (h) | 1.57 | (10.3) | 1.53 | (9.7) | 1.54 | (8.8) |
| CL/F | 31.5 | (16.4) | 37.4 | (16.5) | 30.3 | (16.1) |
| (L/h) | | | | | | |
| V_{z}/F (L) | 70.7 | (13.1) | 82.6 | (18.4) | 66.8 | (12.0) |
| MRT (h) | 2.84 | (13.3) | 3.53 | (9.4) | 2.52 | (19.6) |

| | | | | | |
|---|---|---|---|---|---|
| Fᵣₑₗ | 0.964 | (8.8) | 0.817 | (13.2) | NA |

Arithmetic mean (CV %) data are presented for all parameters except tₘₐₓ
Median (min, max) data are presented for tₘₐₓ
n = number of subjects

Similar relative bioavailability, with respect to AUC_{0-inf}, was observed for the 4×2.5 mg MR film coated tablets compared with the 1×10 mg conventional capsule (estimated ratio=0.959; 95% CIs=0.899, 1.02). Bioavailability was statistically significantly lower, with respect to Cₘₐₓ, for the 4×2.5 mg MR film coated tablets compared with the 1×10 mg conventional capsule (estimated ratio=0.777; 95% CIs=0.687, 0.880). Relative bioavailability was statistically significantly lower, with respect to AUC_{0-inf}, for the 1×10 mg slow MR film coated tablet compared with the 1×10 mg conventional capsule (estimated ratio=0.810; 95% CIs=0.760, 0.864). Bioavailability was also statistically significantly lower, with respect to Cₘₐₓ, for the 1×10 mg slow MR film coated tablet compared with the 1×10 mg conventional capsule (estimated ratio=0.375; 95% CIs=0.331, 0.424). Gaboxadol was rapidly absorbed following the oral administration of a single 10 mg dose, with tₘₐₓ occurring marginally later for both matrix treatments (between 1.0 and 1.5 hours post-dose) compared with the conventional capsule (0.583 hours post-dose). No statistical differences in t^{1/2} were observed between the two matrix treatments and the 1×10 mg conventional capsule, with mean values ranging between 1.53 and 1.57 hours.

### EXAMPLE 5

### Pharmacokinetic Comparison of Gaboxadol 15 mg ODT Formulation to a Gaboxadol Monohydrate 15 mg Capsule Formulation

This was an open-label, randomized, 2-period, single-dose, balanced crossover study in 24 healthy, young adult male and female subjects (at least 6 of each gender). All subjects received 1 of the 2 different treatments in each study period. Treatment A was a single, oral dose of a 15-mg gaboxadol ODT administered (placed on the tongue) in a fasted state without water. Treatment B was a single, oral dose of a 15-mg gaboxadol monohydrate capsule administered in a fasted state with 240 mL of water. Subjects were randomized with respect to treatment order. Following each single oral dose of each formulation, plasma samples for gaboxadol assay were collected up to 16 hours post dose. There was a minimum 4-day washout interval between dosing in each treatment period.

The plasma pharmacokinetic profile (AUC_{0-∞}, Cₘₐₓ, T_{max,} apparent t^{1/2}, Cl/F, V_{z}/F) of each treatment was measured for all subjects. Blood samples for plasma gaboxadol concentration determination were collected through 16 hours following the administration of study drug in each treatment period. Whole blood samples were collected at the protocol-specified time points into sodium heparin Vacutainer polypropylene tubes and processed for analysis for gaboxadol. The samples were slowly mixed by inversion 6 to 8 times and centrifuged at 1500 g for a minimum of 5 minutes at 4° C. The plasma was separated, transferred to round bottom 4.5-mL NUNC polypropylene tubes, and stored frozen at −70° C. Samples were spun and separated within 30 minutes of sampling. The samples were labeled with computer-generated labels.

Cₘₐₓ and Tₘₐₓ were obtained by inspection of the concentration-time data. Actual sampling times were used to determine Tₘₐₓ. AUC to the last time point was calculated using the linear trapezoidal method for ascending concentrations and the log trapezoidal method for descending concentrations. A linear regression was performed on the log-transformed plasma concentration-time data in the apparent elimination phase to obtain the rate constant of elimination (k). The apparent terminal half-life was calculated using the relationship t_{1/2}=1 n(2)/k. AUC_{0-∞}, was estimated as the sum of AUC to the last measured concentration and the extrapolated area given by the quotient of the last measured concentration and k. Cl/F was calculated as the ratio of the dose to AUC_{0-∞} and V_{z}/F was calculated as the ratio of Cl/F to k. AUC, Cₘₐₓ, Cl/F and V_{z}/F were adjusted based on the assay potency of respective tablet or capsule formulation.

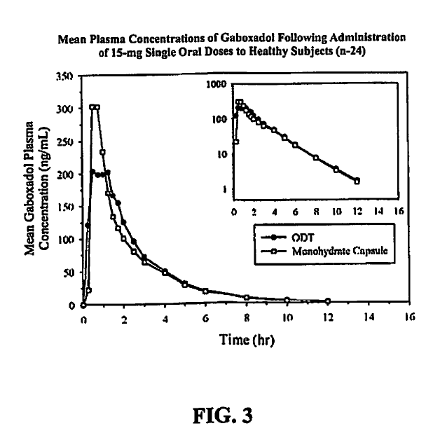

FIG. 3 shows the mean plasma concentrations of gaboxadol following administration of the ODT and monohydrate capsule formulations. TABLE IV summarizes the potency-adjusted plasma pharmacokinetic parameters (adjusted for assayed potencies of the formulations) of gaboxadol following administration of a 15-mg gaboxadol ODT, or a 15-mg gaboxadol monohydrate capsule.

TABLE IV
Summary of Potency-Adjusted Pharmacokinetic Parameters
of GBX Following Administration of 15-mg Single
Oral Doses to Healthy Subjects (n = 24)

| | | | | | Ratio^{‡} of Geometric | | |
|---|---|---|---|---|---|---|---|
| Pharma- | | | | | Means (ODT/Mono- | | |
| cokinetic | Geometric Means^{†} | | | | hydrate Capsule) | | |
| Parameter | | | Monohydrate | | and 90% Confi- | | |
| (units) | ODT | | Capsule | | dence Interval | | MSE |
| AUC₀₋_{∞} | 573 | | 560 | | 1.02 | (1.00, 1.05) | 0.0028 |
| (ng · | | | | | | | |
| hr/mL) | | | | | | | |
| Cₘₐₓ | 336 | | 386 | | 0.87 | (0.77, 0.99) | 0.0645 |
| (ng/mL) | | | | | | | |
| Tₘₐₓ^{§} | 0.75 | | 0.50 | | 0.188 | (0.000, 0.500) | |
| (hr) | | | | | | | |
| Apparent | 1.67 | | 1.64 | | | | |
| t_{1/2}^{§} | | | | | | | |
| (hr) | | | | | | | |
| Cl/F | 443 | (73) | 452 | (75) | | | |
| (mL/min) | | | | | | | |
| (SD) | | | | | | | |
| V_{z}/F (L) | 65 | (11) | 65 | (12) | | | |
| (SD) | | | | | | | |

^{†}AUC₀₋_{∞} and Cₘₐₓ statistics based on least squares estimates from ANOVA performed on natural log-transformed values. Cl/F and V_{z}/F statistics are arithmetic mean and SD (standard deviation), median is shown for Tₘₐₓ, and harmonic mean is shown for apparent terminal t_{1/2}.
^{‡}For Tₘₐₓ, Hodges-Lehmann estimate of the median and 90% CI for treatment difference.
^{§}Not adjusted for potency.
Mean squared error (MSE) from ANOVA model on the natural log scale.

### EXAMPLE 6

### Assessment of Residual Effects Resulting from Gaboxadol Administration

This study was a double blind, double-dummy, randomized, active- and placebo-controlled, single dose, 3-period crossover study, followed by an open-label, single-dose, single period study in healthy elderly male and female subjects. See, Boyle et al., Hum Psychopharmacol. 2009 January; 24 (1):61-71. Subjects were randomized to each of 3 treatments (Treatments A, B, and C) to be administered in a crossover manner over the first 3 treatment periods. For Treatment A, subjects received a single dose of gaboxadol 10 mg; for Treatment B, subjects received a single dose of flurazepam 30 mg; and for Treatment C, subjects received a single dose of placebo. Doses were administered orally at bedtime on Day 1. Subjects were domiciled from early in the evening of dosing until ˜36 hours post-dose (morning of Day 3) during each treatment period. The subjects who participated in treatment periods 1-3 participated in a fourth treatment period. In this period, a single dose of gaboxadol 10 mg (Treatment D) was administered orally in an open-label manner on the morning of Day 1 for PK of gaboxadol. There was at least a 14-day washout between the doses of consecutive treatment periods. Study participants included healthy, elderly male and female subjects between 65 and 80 years of age, with a Mini Mental Status 24, weighing at least 55 kg. All subjects received 10 mg gaboxadol monohydrate capsules and 30 mg flurazepam (provided as 2×15 mg capsules), matching placebo was provided for both gaboxadol and flurazepam.

The primary endpoints evaluated included pharmacodynamics (measurement of psychomotor performance, memory, attention and daytime sleepiness the following pm dosing), gaboxadol pharmacokinetics, and safety. Gaboxadol (single dose 10 mg) did not show residual effect 9 hours post-dose on the primary endpoints Choice Reaction Time and Critical Flicker Fusion, whereas the active reference flurazepam (30 mg single dose) showed significant effect on the same tests. In addition, gaboxadol did not show any signs of residual effects on other measurements applied in the study (Multiple Sleep Latency Test (MSLT); Digit symbol substitution test (DSST), Tracking, Memory tests, Body Sway, and Leeds Sleep Evaluation Questionnaire).

### EXAMPLE 7

### Prospective Assessment of the Efficacy of Gaboxadol in Patients with Angelman syndrome

This study is designed to determine whether gaboxadol leads to an improvement in one or more symptoms of Angelman syndrome. Participants are randomized into 6 separate treatment groups (A-F). Inclusion criteria for randomization require that each participant has been previously diagnosed with Angelman syndrome by clinical evaluation or that the participant is diagnosed with one or more of the major and minor criteria for Angelman syndrome.

Major Criteria include:
Functionally severe developmental delay
Speech impairment; none or minimal words used
Movement or balance disorder
Behavioral uniqueness, frequent laughs/smiling, excitable personality, hand flapping, short attention span

Minor Criteria include:
Deceleration in head circumference growth (post-natal)
Seizures (myoclonic, absence, drop, tonic-clonic)
Abnormal EEG (with patterns suggestive of AS, or hypsarrhythmia)
Sleep disturbance
Attraction to or fascination with water
Drooling

After randomization the participants are placed into 6 separate treatment groups (A-F) and a placebo group. Treatment group A receives 20 mg gaboxadol in the evening. Treatment group B receives 15 mg gaboxadol in the evening. Treatment group C receives 15 mg gaboxadol in the evening and 5 mg gaboxadol in the morning. Treatment group D receives 10 mg gaboxadol in the evening. Treatment group E receives 10 mg gaboxadol in the evening and 10 mg gaboxadol in the morning. Treatment group F receives 10 mg gaboxadol in the evening and 5 mg gaboxadol in the morning.

Participants are assessed throughout the treatment period to determine whether gaboxadol administration leads to an improvement in one or more symptoms of Angelman syndrome. Several behavioral domains; communication, attention, maladaptive behaviors, and hyper-excitability are assessed. To quantify the communication behavior, participants engage in an unstructured play session to elicit speech and non-verbal communication attempts. Speech attempts by the child are transcribed phonetically and categorized into five different types of vocalizations using the Stark Assessment of Early Vocal Development-Revised (SAEVD-R) which categorizes non-speech and pre-speech sounds (protophones), as well as vowels, consonants and syllables.

Gait abnormalities occur in most cases of Angelman syndrome. Thus, five primary spatiotemporal parameters are analyzed: cadence, gait velocity, stride width, step length and percent stance. For each parameter, a principal component analysis is used to establish a gait index for assessment of the subjects.

In addition, primary outcome measures that may be assessed include changes in raw or standard scores between baseline and after trial completion of:
I. Bayley Scales of Infant and Toddler Development, 3rd edition (or the Mullen Scales of Early Learning in the more developmentally advanced subjects);
II. Vineland Adaptive Behavior Scales, 2nd edition (standard scores only);
III. Preschool Language Scale, 4th edition;
IV. Aberrant Behavior Checklist—Community version; and
V. A change from baseline in the Clinical Global Impressions Severity Scale Score.

Secondary outcome measures include normalization of the electroencephalogram (EEG) signature when comparing post gaboxadol administration results to baseline results.

### EXAMPLE 8

### Prospective Assessment of the Efficacy of Gaboxadol in Patients with Angelman Syndrome

This study is designed to determine whether lower doses of gaboxadol lead to an improvement in younger patients or patients with less severe clinically evaluated symptoms. For example, adolescent patients (age 12-18 years) may have the similar clinical presentation and baseline disease characteristics as the adult population but the reduction in ambulation may be less severe. In these patients it is anticipated that the target benefit of gaboxadol will also include the reduction in ataxia and the improvement in ambulatory function.

In pediatric patients (6 months to 12 years) the diagnosis of Angelman Syndrome is usually made around 1 year of age based on important delay in the development status and eventually persistent seizures. As the child grows older, additional neurologic deficit will contribute to the disease presentation leading to ataxia and walking disability. For these prospective participants, the inclusion criteria for randomization and assessment procedures is similar to that previously described.

After randomization the participants are placed into 6 separate treatment groups (A-F) and a placebo group. Treatment group A receives 7.5 mg gaboxadol in the evening. Treatment group B receives 5 mg gaboxadol in the evening. Treatment group C receives 5 mg gaboxadol in the evening and 2.5 mg gaboxadol in the morning. Treatment group D receives 2.5 mg gaboxadol in the evening. Treatment group E receives 2.5 mg gaboxadol in the evening and 1 mg gaboxadol in the morning. Treatment group F receives 1 mg gaboxadol in the evening.

Participants are assessed in accordance with parameters set forth in above Example 7.

### EXAMPLE 9

### Prospective Assessment of the Efficacy of Gaboxadol in Patients with Fragile X Syndrome

This study is designed to determine whether gaboxadol leads to an improvement in one or more symptoms of Fragile X syndrome. Participants are randomized into 6 separate treatment groups (A-F). Inclusion criteria for randomization require patients that have been diagnosed with Fragile X syndrome. For example, patients who are at least moderately ill based on a Clinical Global Impression Severity score of at least 4 and have qualifying scores on the ABC-C and IQ test.

After randomization the participants are separated into 6 treatment groups (A-F) and a placebo group. Treatment group A receives 20 mg gaboxadol in the evening. Treatment group B receives 15 mg gaboxadol in the evening. Treatment group C receives 15 mg gaboxadol in the evening and 5 mg gaboxadol in the morning. Treatment group D receives 10 mg gaboxadol in the evening. Treatment group E receives 10 mg gaboxadol in the evening and 10 mg gaboxadol in the morning. Treatment group F receives 10 mg gaboxadol in the evening and 5 mg gaboxadol in the morning.

Participants are assessed throughout the treatment period to determine whether administration of gaboxadol leads to an improvement in one or more symptoms of Fragile X syndrome. In particular, patients are assessed using one or more primary and secondary outcome measures. Primary Outcome Measures may include:

Change From Baseline in Behavioral Symptoms of Fragile X Syndrome Using the Aberrant Behavior Checklist-Community Edition (ABC-CFX) Total Score;

Global Improvement of Symptoms in Fragile X Using the Clinical Global Impression- Improvement (CGI-I) Scale;

Change From Baseline in Irritability, Lethargy/Withdrawal, Stereotypic Behavior, Hyperactivity, Inappropriate Speech and Social Avoidance Assessed by the Individual Subscales of the ABC-CFX Scale;

Change From Baseline in Repetitive Behaviors Assessed Using the Repetitive Behavior Scale—Revised (RBS-R) Scores;

Visual Analogue Scale (Behavior); Expressive Vocabulary Test; Vineland Adaptive Behavior Scale-II (VABS-II) Adaptive Behavior Composite Score; and Aberrant Behavior Checklist-Community Edition (ABC-C) Composite Score.

### EXAMPLE 10

### Prospective Assessment of the Efficacy of Gaboxadol in Patients with Fragile X Syndrome

This study is designed to determine whether lower doses of gaboxadol lead to an improvement in younger patients or patients with less severe clinically evaluated symptoms. For these participants, the inclusion criteria for randomization and assessment procedures is similar to that previously described.

After randomization the participants are randomized into 6 separate treatment groups (A-F) and a placebo group. Treatment group A receives 7.5 mg gaboxadol in the evening. Treatment group B receives 5 mg gaboxadol in the evening. Treatment group C receives 5 mg gaboxadol in the evening and 2.5 mg gaboxadol in the morning. Treatment group D receives 2.5 mg gaboxadol in the evening. Treatment group E receives 2.5 mg gaboxadol in the evening and 1 mg gaboxadol in the morning. Treatment group F receives 1 mg gaboxadol in the evening.

Participants are assessed throughout the treatment period to determine whether administration of gaboxadol leads to an improvement in one or more symptoms of Fragile X syndrome. In particular, patients are assessed using one or more primary and secondary outcome measures. Primary Outcome Measures may include:

Change From Baseline in Behavioral Symptoms of Fragile X Syndrome Using the Aberrant Behavior Checklist-Community Edition (ABC-CFX) Total Score;

Global Improvement of Symptoms in Fragile X Using the Clinical Global Impression- Improvement (CGI-I) Scale;

Change From Baseline in Irritability, Lethargy/Withdrawal, Stereotypic Behavior, Hyperactivity, Inappropriate Speech and Social Avoidance Assessed by the Individual Subscales of the ABC-CFX Scale;

Change From Baseline in Repetitive Behaviors Assessed Using the Repetitive Behavior Scale—Revised (RBS-R) Scores;

Visual Analogue Scale (Behavior); Expressive Vocabulary Test; Vineland Adaptive Behavior Scale-II (VABS-II) Adaptive Behavior Composite Score; and Aberrant Behavior Checklist-Community Edition (ABC-C) Composite Score.

### EXAMPLE 11

### Prospective Assessment of the Efficacy of Gaboxadol in Patients with Fragile X-Associated Tremor/Ataxia Syndrome

This study is designed to determine whether gaboxadol leads to an improvement in one or more cognitive symptoms of Fragile X-associated tremor/ataxia syndrome (FXTAS) and involves a placebo-controlled, double-blind, randomized clinical trial. Participants will be individuals with FXTAS. FMR1 CGG repeat lengths will be quantified in all subjects using conventional procedures. FXTAS will be diagnosed following published criteria (Bacalman et al., Clin Psychiatry 2006, 67:87-94; Jacquemont et al., Lancet Neurol 2003, 6:45-55). For the main gaboxadol trial, 200 will be screened for eligibilty. Randomization to either placebo or gaboxadol will be blinded to all study personnel, investigators, and participants until the end of the one year trial period. Participants will participate in a word repetition/event related potentials (ERPs) experiment.

Identical appearing tablets containing either gaboxadol or placebo will be administered. After randomization the participants are randomized into 6 separate treatment groups (A-F) and a placebo group. Treatment group A receives 7.5 mg gaboxadol in the evening. Treatment group B receives 5 mg gaboxadol in the evening. Treatment group C receives 5 mg gaboxadol in the evening and 2.5 mg gaboxadol in the morning. Treatment group D receives 2.5 mg gaboxadol in the evening. Treatment group E receives 2.5 mg gaboxadol in the evening and 1 mg gaboxadol in the morning. Treatment group F receives 1 mg gaboxadol in the evening.

Neuropsychological testing will involve assessing each participant's verbal memory with the California Verbal Learning Test (CVLT). Executive functioning will be evaluated with the Behavioral Dyscontrol Scale (BDS, a well-validated nine-item test measuring the intentional control of simple voluntary motor behavior) and the Controlled Oral Word Association Test (COWAT). Word repetition ERP experiments will involve electroencephalogram (EEG) and ERP recordings during a semantic category decision/word repetition task to be performed at both the baseline visit and the 1-year follow-up. Participants will be fit with an electrode cap and seated 100 cm from a video monitor. Category statements will be read aloud by an experimenter, each followed (˜1 s later) by a target word visually presented in the center of the monitor (duration=300 ms, visual)angle=0.4°). Participants are not instructed to not respond or move for 3 seconds following the target word, then to say the word seen and a ‘yes’ or ‘no’ judgment indicating whether the word fit with the preceding category statement (congruous trial) or not (incongruous trial). EEG sessions will consist of three blocks of 144 trials, each block lasting slightly over 20 min. Stimuli will be 216 spoken phrases each describing a category (e.g., ‘a body of water’), and followed by an associated target word. Categories and target words will be selected with the aid of published norms and local norms based on administered questionnaires. Half of the targets (congruous words) will be medium-typicality category exemplars (e.g., ‘river’ for ‘a body of water’). The other half of the targets, will be matched to congruous targets in length and word frequency, and will be concrete nouns that are incongruous with their preceding categories (e.g., ‘visitor’ for ‘part of a watch’).

Participants will be randomly assigned to one of three counterbalanced stimulus lists, which includes 36 congruous category-target pairs presented once, 36 presented twice, 36 presented three times, and equal numbers of incongruous pairs in the same repetition manners, giving a total of 432 trials. Therefore, half of the stimuli are congruous and half are incongruous, and half are new, whereas half are repeats. Repeated targets will always follow the same category statement as in the initial presentation. For category-target pairs repeated once, the lag between the first and second presentations will be 0-3 intervening trials (spanning 10-40 s). For pairs repeated twice, the lag for both second and third presentations will be 10-13 intervening trials (˜100-140 s). Electrophysiological recording involves using thirty two (32)-channel EEG. Tin electrodes embedded in an elastic electrode cap will be placed at midline (Fz, Cz, Pz, POz), lateral frontal (F3/4, F7/8, FC1/2, FP1/2), temporal (T5/6), parietal (P3/4, CP1/2), and occipital sites (O1/2, PO7/8), defined by the International 10-20 System. Additional electrodes include bilateral pairs approximating Broca's area (BL), Wernicke's area (WL), and their right hemisphere homologs (BR and WR, respectively), and an electrode pair at 33% of the interaural distance lateral to Cz over the superior temporal lobe (L41, R41). Inter-electrode impedance will be maintained below 5 kΩ. All scalp electrodes will be referenced online to the left mastoid and re-referenced offline to the average of both mastoids. Eye movements will be monitored by electrooculogram (EOG) recording with four electrodes (one beneath and one at the outer canthus of each eye). The EEG will be amplified and digitized by a Nicolet SM 2000 amplifier with bandpass of 0.016-100 Hz, and a sampling rate of 250 Hz. Subsequent memory tests for target words will involve, immediately following the EEG recording, three unanticipated subsequent memory tests (free recall, cued-recall, and multiple-choice recognition) to be administered in that order. In the free recall task, participants will be asked to write down as many target words seen in the ERP experiment as they can remember. A list of 40 written category statements (22 from congruous trials and 18 from incongruous trials) will be provided in the cued-recall test, and subjects will be instructed to fill in the target word presented after a given category statement, or to provide the ‘first word that comes to mind’ if they cannot recall the associated target word. The multiple-choice recognition task consists of the same 40 category statements, each followed by the associated target word and five foils never presented during the experiment.

The treatment effects on subsequent memory performance changes (1 year-baseline) will be examined by ANCOVA with the within-subject factor of visit, between-subjects factor of treatment, and covariate of gender (because FXTAS is an X-chromosome-related disorder and there are well-established gender differences in the clinical expression of FXTAS. Continuous EEG data will be segmented into epochs of 1024 ms duration (starting at 100 ms before the target word onset until 924 ms poststimulus onset). Epochs contaminated with artifacts such as blinks, horizontal eye movements, excessive muscle activity, or amplifier blocking will be rejected from further data analyses (mean rejection rate=49.1%, no differences between groups or visits, p's>0.75). Artifact-free epochs will be averaged by experimental condition to produce ERP waveforms. Mean amplitude (within 300-550 ms time window) and 50% fractional area latency of the N400 component will be quantified, for four conditions separately (ie, congruous new/old words, incongruous new/old words). To reduce inter-individual variability, the analyses will focus on two experimental effects: the N400 congruity effect (ERPs to incongruous new words minus congruous new words), and the N400 repetition effect (ERPs to incongruous new words minus incongruous old words). The P600 repetition effect will also be calculated by subtracting ERPs to congruous old words from congruous new words in the 550-800 ms time window. Repeated-measures ANOVA with the between-subjects factor of treatment, and within-subjects factors of visit and electrode will be performed on N400 and P600 data with 26 scalp electrodes (excluding FP1/2). The Greenhouse-Geisser correction will be used to correct for violations of sphericity, and adjustedp-values of<0.05 will be considered significant. Pearson correlations will be tested between the subsequent memory changes (1 year-baseline) and the N400 repetition effect changes (baseline-1 year). Because the N400 is a negative component, reversing the direction of subtraction will make positive values represent increased N400 repetition effect amplitude after 1 year. Composite N400 amplitude and latency measures averaged from a right posterior cluster of five electrodes (P4, T6, WR, 02, and P08) will be used in correlational analyses.

### EXAMPLE 12

### Prospective Assessment of the Efficacy of Gaboxadol in Patients with Fragile X-Associated Tremor/Ataxia Syndrome

This study is designed to determine whether gaboxadol leads to an improvement in cognitive symptoms, i.e., attentional processes which are fundamental to executive function/dysfunction associated with Fragile X-associated tremor/ataxia syndrome (FXTAS) and involves a placebo-controlled, double-blind, randomized clinical trial and an auditory “oddball” task. Participants will be individuals with FXTAS. FMR1 CGG repeat lengths will be quantified in all subjects using conventional procedures. FXTAS will be diagnosed following published criteria (Bacalman et al., Clin Psychiatry 2006, 67:87-94; Jacquemont et al., Lancet Neurol 2003, 6:45-55). For the main gaboxadol trial, 200 potential participants will be screened for eligibility. Randomization to either placebo or gaboxadol will be blinded to all study personnel, investigators, and participants until the end of the one year trial period. Participants will participate in an auditory “oddball”/event related potentials (ERPs) experiment.

Identical appearing tablets containing either gaboxadol or placebo will be administered. After randomization the participants are randomized into 6 separate treatment groups (A-F) and a placebo group. Treatment group A receives 7.5 mg gaboxadol in the evening. Treatment group B receives 5 mg gaboxadol in the evening. Treatment group C receives 5 mg gaboxadol in the evening and 2.5 mg gaboxadol in the morning. Treatment group D receives 2.5 mg gaboxadol in the evening. Treatment group E receives 2.5 mg gaboxadol in the evening and 1 mg gaboxadol in the morning. Treatment group F receives 1 mg gaboxadol in the evening.

In the auditory “oddball” experiment, patients will be instructed to detect an infrequent “oddball” tone embedded in a train of non-target standard tones. Subjects will press a button to each target detected and also keep a mental count of the number of targets in that experimental block. Prior studies in premutation carriers using the same “oddball” paradigm have demonstrated an altered frontal P300 (P3) ERP component in FXTAS patients, which tracks their executive dysfunction. See, Yang et al., Ann Neurol 74, 275-283 (2013); Yang et al., Cereb Cortex 23, 2657-2666 (2013). In these studies and others, the earlier abnormalities of prolonged N100 latency and reduced P200 (P2) amplitude were also found in a predominately male FXTAS group but not in female premutation carriers asymptomatic of FXTAS9.

Neuropsychological testing will involve examining each patient's EEG. Accordingly, EEG during a two-stimulus auditory oddball experiment will be recorded in a sound-attenuated, dimly-lit chamber. Lower (113 Hz) and higher (200 Hz) frequency pure tones will be presented at 40 dB above individual hearing level in 4 blocks, each containing 100 tones, with a stimulus onset asynchrony jittered from 1.0-1.5 seconds. Prior to each block, subjects will be instructed to respond to the infrequent (probability equaling 25%) “oddball” tones (high or low target tones, counterbalanced across blocks). A dual task will be employed in which subjects are instructed to press a button to each target tone, and to also keep a mental count of the number of targets in each block. The mental count of target tones will be reported immediately following completion after each block. 32-channel EEG will be recorded with a Nicolet-SM-2000 amplifier (band-pass=0.016-100 Hz, sampled at 250 Hz). Data Analysis will involve the |count-hit| discrepancy in each block (i.e., the absolute value of the difference between correct button-presses and mental count to target tones within a block) will be calculated for each participant, as an inverse measure (i.e., a lower value represents better performance) of attention/working memory performance during the oddball task. Event-locked EEG segments contaminated with blinks, eye movements, excessive muscle activity, or amplifier blocking will be rejected using a semi-automated computer algorithm. Artifact-free EEG segments of 1024 ms (with a 100 ms pre-stimulus baseline period, and 924 ms post-stimulus onset) will be averaged by experimental condition to obtain the ERPs. Mean amplitude and local peak latency of 4 ERP components will be quantified in the following time windows: N100 (N1, 70-150 ms), P2 (160-260 ms), N200 (N2, 170-300 ms), and P3 (300-650 ms). The waveforms to both target and standard tones will be used to measure N1. The P2 will be measured from ERPs to standard tones. The N2 component is defined from the difference wave (ERPs to targets minus standards). The P3 will be measured from both the difference wave and the ERP waveform to targets. ERP measures will be submitted to repeated-measures ANOVAs (SPSS 22, IBM) with the between-subjects factor of treatment, and the within-subjects factors of visit and electrode. Analyses of N1 and P2 will include 4 fronto-central electrodes (Fz, Cz, FC1/2). Five central channels (Cz, FC1/2, CP1/2) will be used for the N2 analyses. P3 analyses will be carried out with 26 scalp electrodes (all except FP1/2). The Greenhouse-Geiser correction will be used to adjust for violations of sphericity, where appropriate. To further characterize the modulatory effects of gaboxadol on the P2 component, a habituation analysis will be conducted for P2 amplitude. P2 mean amplitude in response to the first 30 standard tones will be compared to the amplitude of response to the last 30 standard tones within the first block of each study, with the between-subjects factor of treatment, and the within-subjects factors of visit, trial position, and electrode. Data from a group of 16 age-matched normal controls, each of whom will have only underwent one ERP recording, will be used to demonstrate the normal habituation effect. Linear regression will be used to examine the correlations between changes (1-year follow-up minus baseline) in the (count-hitl discrepancy and in ERP measures for which significant treatment effects are shown. Correlations between local peak amplitudes of P2 (measured after application of a 30 Hz low-pass filter) and CGG repeats will be tested.

### EXAMPLE 13

### Prospective Assessment of the Effect of Gaboxadol on preCGG Knock-In Mice Hippocampal Neurons

Knock-in (KI) mouse models have been developed as models of Fragile X-associated tremor ataxia. In one mouse model, a native 9-10 CGG repeat allele in the homologous Fmr1 gene was replaced with CGG expansion repeats that can vary from 100 to >300 in size from generation to generation. See, Berman, R. F. and Willemsen, R. Mouse models of Fragile x-associated tremor ataxia, J. Investig. Med., (2009) 57, 837-841. Another K1 mouse model was developed wherein CGG-CCG repeats were serially ligated in exon 1 of the endogenous mouse Fmr1 gene. See, Entezam, et al., Gene, (2007) 395, 125-134. Similar to human premutation carriers, the hippocampus of premutation mice exhibits elevated Fmr1 mRNA and normal to 50% reductions in FMRP compared with wild-type (WT), even in mice with large (150-190) repeats. See, Cao et al., Human Molecular Genetics, (2012) 21:13, 2923-2935. Although the premutation mouse models do not fully recapitulate human FXTAS, they do show progressive deficits in processing spatial and temporal information, cognitive deficits, motor deficits and hyperactivity. Id.

All preCGG KI and WT mice in the C57 B6 background will be housed under standard vivarium conditions. PreCGG hemizygous male mice (150-190 CGG expansion repeat; average 170) will be obtained by breeding homozygous preCGG females with preCGG hemizygous males. Male WT and preCGG pups delivered on the same day will be used for paired cultures. The FMR1 genotype will be verified by PCR Genotyping will be accomplished by extracting DNA from mouse tail, and genotyping of the TMR1 expansion size will be performed using known forward and reverse primers, Hippocampal neuron-astrocyte co-culture will be obtained from the brains of postnatal day 0-1 (P0-1) WT or preCGG KI male pups. Hippocampal neurons will be dissociated and plated onto poly-L-lysine-coated 6-well plates or clear-bottom, black well, 96-well imaging plates (BD, Franklin Lakes, N.J., USA) at densities of 2×106/well or 1×105/well, respectively. For microelectrode array (MEA) experiments, 60 ml of cell suspension at a density of 2×106 cells/ml will be added as a drop to the center of MEA to cover the 64 electrode probes. After 2 h incubation, a volume of 1.5 ml of serum-free neurobasal supplemented medium containing NS21 supplement, 0.5 mM L-glutamine and HEPES will be added to each MEA. The medium will be changed twice a week by replacing half volume of culture medium with serum-free neurobasal supplemented medium. The cells will be maintained. in an atmosphere at 378 C with 5% CO2 and 95% humidity. Enriched hippocampal astrocytes will be obtained from P0-1 WT or preCGG KI male pups. The dissection and dissociation of the cells is the same as the neuronal preparation. After digestion and centrifugation as described for the isolation of neurons, cells will be suspended in DMEM medium supplemented with 10% fetal bovine serum, 100 IU/ml penicillin and 0.10 mg/ml streptomycin, pH 7.4. Cells will be plated onto poly-L-ornithine-coated T-75 culture flask at a density of 2-3×106 cells/flask and maintained in an incubator at 378 C with 5% CO2 and 95% humidity. The culture medium will be changed twice a week and the cells will be used between 2 and 4 weeks and no more than three passages.

Quantitative measurements of Fmr1 mRNA levels will involve isolation of total RNA from primary hippocampal cultures by a standard method (Trizol, Ambion, Inc., Austin, Tex., USA). Precise estimates of Fmr1 mRNA levels in total RNA will be obtained by real-time PCR. Details of the method and its application to the study of Fmr1 mRNAs are as described previously. See, Tassone et al., Am. J. Hum. Genet. (2000) 66, 6-15. The reference gene can be b-glucoronidase (GUS). The analysis will be repeated for three different RNA concentrations, in duplicate, and incorporated standards for each determination to compensate for any changes in reaction efficiency.

All MEA recordings will be performed at 378 C in culture medium without perfusion with the MED64 system (Alpha MED Scientific, Inc., Ibaraki, Osaka, Japan). MEA probes will be loaded in the MED-C03 chamber and the raw data will be acquired using the Mobius software (Alpha MED Scientific, Inc., Ibaraki, Osaka, Japan). Signals from the amplifier can be digitized at a rate of 20 kHz and using a high-pass filter (cutoff frequency of 100 Hz). The Mobius software can be used to detect spontaneous events that exceeded a threshold of 225 mV. The time stamps for each channel will then be saved and exported to an Excel sheet. For raster plot as well as spike rate and burst analysis, data can be imported into NeuroExplorer software (version 4.0, NEX Technologies, Littleton, Mass., USA) and analyzed using the raster or burst firing analysis function. For an activity to be defined as a burst, spikes should occur within 350 ms of each other. In addition, a burst may consist of a minimum of four spikes with minimum burst duration of 100 ms. The minimum separation between bursts will he set at 350 ms. Using this measurement, the burst duration will be measured from the start of a burst until the gap to the next spike is more than 350 ms, so one repetitive burst may include multiple high-frequency bursts.

Graphing and statistical analysis can be performed using the GraphPad Prism software (Version 5.0, GraphPad Software, Inc., San Diego, Calif., USA). Statistical significance between different groups can be calculated using Student's t-test or by an ANOVA and, where appropriate, a Dunnett's multiple comparison test. The P-values below 0.05% will be considered significant. Kinetic parameters (Km and Vmax) for Glu uptake can be determined by nonlinear regression analysis of the saturation curves, using the MichaelisMenten equation

Accordingly, the effect of gaboxadol on the electric firing pattern of preCGG hippocampal neurons is evaluated. Electrical firing activity in cultures isolated from WT and preCGG hippocampus will be measured using the 64-electrode multielectrode arrays (MEAs) by converting electrical field potential recordings to raster plots. By culture day 7 DIV, both WI and preCGG hippocampal neurons should display similar patterns of spontaneous electric activity comprising infrequent synchronized bursts of field potentials, mixed with desynchronized random spiking. Neither spike frequency nor burst duration is expected to differ between WI and preCGG cultures at 7 DIV. Synchronized firing activity should increase in frequency and duration as neuronal networks mature by 21 DIV. At 21 DIV, preCGG hippocampal neurons can display a distinct firing pattern composed of intense CBs interspersed with brief periods of quiescence. The percentage of MEAs reporting the CB firing pattern should be significantly higher in the preCGG neuronal cultures than in WI neurons. Micromolar concentrations of gaboxadol, e.g., 0.01 to 10 μM, will be added starting at day 21 DIV and normalization of preCGG hippocampal neurons will be monitored.

While embodiments of the disclosure have been described and exemplified herein, it is not intended that the disclosure be limited thereto, as it is intended that the disclosure be as broad in scope as the art will allow and that the specification be read likewise. Therefore, the above description should not be construed as limiting, but merely as exemplifications of various embodiments. Those skilled in the art will envision other modifications within the scope and spirit of the claims appended hereto.

What is claimed is:

1. A method of treating a disorder selected from the group consisting of Angelman syndrome, Fragile X syndrome and Fragile X-associated tremor/ataxia syndrome comprising administering to a patient in need thereof a pharmaceutical composition comprising about 0.05 mg to about 100 mg gaboxadol or a pharmaceutically acceptable salt thereof wherein the composition provides a Tₘₐₓ of less than 20 minutes.

2. The method of treating a disorder selected from the group consisting of Angelman syndrome, Fragile X syndrome and Fragile X-associated tremor/ataxia syndrome according to claim 1, wherein the composition is a modified release dosage form.

3. The method of treating a disorder selected from the group consisting of Angelman syndrome, Fragile X syndrome and Fragile X-associated tremor/ataxia syndrome according to claim 2, wherein the modified release dosage form comprises an orally disintegrating dosage form.

4. The method of treating a disorder selected from the group consisting of Angelman syndrome, Fragile X syndrome and Fragile X-associated tremor/ataxia syndrome according to claim 2, wherein the modified release dosage form comprises an extended release dosage form.

5. The method of treating a disorder selected from the group consisting of Angelman syndrome, Fragile X syndrome and Fragile X-associated tremor/ataxia syndrome according to claim 2, wherein the modified release dosage form comprises a delayed release dosage form.

6. The method of treating a disorder selected from the group consisting of Angelman syndrome, Fragile X syndrome and Fragile X-associated tremor/ataxia syndrome according to claim 2, wherein the modified release dosage form comprises a pulsatile release dosage form.

7. The method of treating a disorder selected from the group consisting of Angelman syndrome, Fragile X syndrome and Fragile X-associated tremor/ataxia syndrome according to claim 1, wherein the composition comprises 15 mg gaboxadol or a pharmaceutically acceptable salt thereof.

8. The method of treating a disorder selected from the group consisting of Angelman syndrome, Fragile X syndrome and Fragile X-associated tremor/ataxia syndrome according to claim 1, wherein the composition comprises 10 mg gaboxadol or a pharmaceutically acceptable salt thereof.

9. The method of treating a disorder selected from the group consisting of Angelman syndrome, Fragile X syndrome and Fragile X-associated tremor/ataxia syndrome according to claim 1, wherein the composition comprises 5 mg gaboxadol or a pharmaceutically acceptable salt thereof.

10. The method of treating a disorder selected from the group consisting of Angelman syndrome, Fragile X syndrome and Fragile X-associated tremor/ataxia syndrome according to claim 1, wherein the composition provides a Cₘₐₓ of less than 400 ng/ml.

11. The method of treating a disorder selected from the group consisting of Angelman syndrome, Fragile X syndrome and Fragile X-associated tremor/ataxia syndrome according to claim 2, wherein the amount of gaboxadol or a pharmaceutically acceptable salt thereof within the patient 4 hours after administration of the pharmaceutical composition is between about 65% to about 85% less than the administered dose.

12. The method of treating a disorder selected from the group consisting of Angelman syndrome, Fragile X syndrome and Fragile X-associated tremor/ataxia syndrome according to claim 2, wherein the amount of gaboxadol or a pharmaceutically acceptable salt thereof within the patient 4 hours after administration of the pharmaceutical composition is more than 50% of the administered dose.

13. The method of treating a disorder selected from the group consisting of Angelman syndrome, Fragile X syndrome and Fragile X-associated tremor/ataxia syndrome according to claim 1 wherein the composition provides improvement in at least one symptom selected from the group consisting of tremors, rigidity, ataxia, bradykinesia, gait, speech impairment, vocalization difficulties, cognition impairment, impaired motor activity, clinical seizure, hypotonia, hypertonia, feeding difficulty, drooling, mouthing behavior, sleep difficulties, hand flapping, easily provoked laughter, short attention span, reduced sensation, numbness or tingling, pain, muscle weakness in the lower limbs, inability to control the bladder or bowel, chronic pain syndromes, fibromyalgia, migraine, hypothyroidism, hypertension, sleep apnea, vertigo, olfactory dysfunction, hearing loss, short-term memory loss, loss of executive function, impulse control difficulties, self- monitoring difficulties, attention focusing difficulties, cognitive inflexibility, anxiety, depression, moodiness, irritability.

14. A pharmaceutical dosage form comprising: a) a therapeutically effective amount of gaboxadol or a pharmaceutically acceptable salt thereof; and b) at least one pharmaceutically acceptable excipient, wherein the dosage form provides a Tₘₐₓ of less than about 20 minutes and a Cₘₐₓ greater than about 50 ng/mL.

15. The pharmaceutical dosage form according to claim 14, wherein the Cₘₐₓ is less than about 400 ng/ml.

16. The pharmaceutical dosage form according to claim 14, wherein the dosage form is a modified release dosage form.

17. The pharmaceutical dosage form according to claim 16, wherein the modified release dosage form comprises an orally disintegrating dosage form.

18. The pharmaceutical dosage form according to claim 16, wherein the modified release dosage form comprises an extended release dosage form.

19. The pharmaceutical dosage form according to claim 16, wherein the modified release dosage form comprises a delayed release dosage form.

20. The pharmaceutical dosage form according to claim 16, wherein the modified release dosage form comprises a pulsatile release dosage form.

21. The pharmaceutical dosage form according to claim 14, wherein the amount of gaboxadol or a pharmaceutically acceptable salt thereof ranges from 0.05 mg to 100 mg.

22. The pharmaceutical dosage form according to claim 21, wherein the dosage form provides delivery of gab oxadol or a pharmaceutically acceptable salt thereof such that the amount of gaboxadol or a pharmaceutically acceptable salt thereof within the patient 4 hours after administration of the pharmaceutical composition is between about 65% to about 85% less than the administered dose.

23. The pharmaceutical dosage form according to claim 21, wherein the dosage form provides delivery of gab oxadol or a pharmaceutically acceptable salt thereof such that the amount of gaboxadol or pharmaceutically acceptable salt thereof within the patient 4 hours after administration of the pharmaceutical composition is more than 50% of the administered dose.
